SEQUENCE LISTING (45 sequences)

SEQ ID NO: 1 - protein, 16 aa, Artificial
  Synthetic construct
RSGYSSPGSPGTPGSR

SEQ ID NO: 2 - protein, 22 aa, Artificial
  Synthetic construct
SGDRSGYSSPGSPGTPGSRSRT

SEQ ID NO: 3 - protein, 11 aa, Artificial
  Synthetic construct
KTHPHFVIPYR

SEQ ID NO: 4 - protein, 16 aa, Artificial
  Synthetic construct
QAKTHPHFVIPYRALV

SEQ ID NO: 5 - protein, 17 aa, Artificial
  Synthetic construct
KYLETPGDENEHAHFQK

SEQ ID NO: 6 - protein, 23 aa, Artificial
  Synthetic construct
AVDKYLETPGDENEHAHFQKAKE

SEQ ID NO: 7 - protein, 12 aa, Artificial
  Synthetic construct
RHDSGYEVHHQK

SEQ ID NO: 8 - protein, 18 aa, Artificial
  Synthetic construct
AEFRHDSGYEVHHQKLVF

SEQ ID NO: 9 - protein, 13 aa, Artificial
  Synthetic construct
KLVFFAEDVGSNK

SEQ ID NO: 10 - protein, 19 aa, Artificial
  Synthetic construct
HHQKLVFFAEDVGSNKGAI

SEQ ID NO: 11 - protein, 12 aa, Artificial
  Synthetic construct
LVFFAEDVGSNK

SEQ ID NO: 12 - protein, 15 aa, Artificial
  Synthetic construct
SGYSSPGSPGTPGSR

SEQ ID NO: 13 - protein, 15 aa, Artificial
  Synthetic construct
RSGYSSPGSPGTPGS

SEQ ID NO: 14 - protein, 18 aa, Artificial
  Synthetic construct
SGDRSGYSSPGSPGTPGS

SEQ ID NO: 15 - protein, 17 aa, Artificial
  Synthetic construct
RSGYSSPGSPGTPGSRS

SEQ ID NO: 16 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 17 - protein, 19 aa, Artificial
  Synthetic construct
GSNKGAIIGLMVGGVVIAT

SEQ ID NO: 18 - protein, 18 aa, Artificial
  Synthetic construct
GSNKGAIIGLMVGGVVIA

SEQ ID NO: 19 - protein, 16 aa, Artificial
  Synthetic construct
GSNKGAIIGLMVGGVV

SEQ ID NO: 20 - protein, 14 aa, Artificial
  Synthetic construct
GSNKGAIIGLMVGG

SEQ ID NO: 21 - protein, 18 aa, Artificial
  Synthetic construct
PLSKAYQGVAAPFPKARR

SEQ ID NO: 22 - protein, 24 aa, Artificial
  Synthetic construct
RGARNSEPQDEGELFQGVDPRALA

SEQ ID NO: 23 - protein, 24 aa, Artificial
  Synthetic construct
GAYKAIPVAQDLNAPSDWDSRGKD

SEQ ID NO: 24 - protein, 16 aa, Artificial
  Synthetic construct
YDGRGDSVVYGLRSKS

SEQ ID NO: 25 - protein, 17 aa, Artificial
  Synthetic construct
SDGRSYESMCEYQRAKC

SEQ ID NO: 26 - protein, 22 aa, Artificial
  Synthetic construct
ECLRCPCSAVTSTGSCSIKSSE

SEQ ID NO: 27 - protein, 26 aa, Artificial
  Synthetic construct
CNNRSASCDALTGACLNCQENSKGNH

SEQ ID NO: 28 - protein, 16 aa, Artificial
  Synthetic construct
ERRKSHEAEVLKQLAE

SEQ ID NO: 29 - protein, 25 aa, Artificial
  Synthetic construct
DDKKGIVDQSQQAYQEAFEISKKEM

SEQ ID NO: 30 - protein, 21 aa, Artificial
  Synthetic construct
ACMKSVTEQGAELSNEERNLL

SEQ ID NO: 31 - protein, 9 aa, Artificial
  Synthetic construct
KGAIIGLMV

SEQ ID NO: 32 - protein, 12 aa, Artificial
  Synthetic construct
KAYQGVAAPFPK

SEQ ID NO: 33 - protein, 18 aa, Artificial
  Synthetic construct
RNSEPQDEGELFQGVDPR

SEQ ID NO: 34 - protein, 17 aa, Artificial
  Synthetic construct
RNSEPQDEGELFQGVDP

SEQ ID NO: 35 - protein, 18 aa, Artificial
  Synthetic construct
KAIPVAQDLNAPSDWDSR

SEQ ID NO: 36 - protein, 10 aa, Artificial
  Synthetic construct
RGDSVVYGLR

SEQ ID NO: 37 - protein, 11 aa, Artificial
  Synthetic construct
RSYESMCEYQR

SEQ ID NO: 38 - protein, 16 aa, Artificial
  Synthetic construct
RCPCSAVTSTGSCSIK

SEQ ID NO: 39 - protein, 20 aa, Artificial
  Synthetic construct
RSASCDALTGACLNCQENSK

SEQ ID NO: 40 - protein, 9 aa, Artificial
  Synthetic construct
KSHEAEVLK

SEQ ID NO: 41 - protein, 20 aa, Artificial
  Synthetic construct
KGIVDQSQQAYQEAFEISKK

SEQ ID NO: 42 - protein, 15 aa, Artificial
  Synthetic construct
KSVTEQGAELSNEER

SEQ ID NO: 43 - protein, 20 aa, Artificial
  Synthetic construct
SGDRSGYSSPGSPGTPGSRS

SEQ ID NO: 44 - protein, 19 aa, Artificial
  Synthetic construct
RSGYSSPGSPGTPGSRSRT

SEQ ID NO: 45 - protein, 6 aa, Artificial
  Synthetic construct
HHHHHH

(19) United States
(12) Patent Application Publication
(10) Pub. No.: US 2021/0262006 A1
(43) Pub. Date: Aug. 26, 2021

(54) Parallel Enzyme Digestion for Protein Biomarker Detection

(71) Applicant: Emory University, Atlanta, GA (US)

(72) Inventors: Nicholas Seyfried, Atlanta, GA (US); Duc Duong, Decatur, GA (US); Allan Levey, Atlanta, GA (US); Maotian Zhou, Atlanta, GA (US); James Lah, Avondale Estates, GA (US)

(21) Appl. No.: 17/254,032

(22) PCT Filed: Jun. 18, 2019
(86) PCT No.: PCT/US2019/037788

§ 371 Date: Dec. 18, 2020

Related U.S. Application Data
(60) Provisional application No. 62/686,385, filed on Jun. 18, 2018.

(51) Int. Cl. C12Q 1/37; G01N 33/68; C07K 14/47

(52) U.S. Cl. CPC C12Q 1/37; G01N 33/6848; G01N 2800/2814; C07K 14/4711; G01N 2800/2821; G01N 33/6896

(57) ABSTRACT

This disclosure relates to isotopically labeled internal standards that can be digested by an enzyme capable of C-terminal cleavage of a first amino acid and/or an enzyme capable of N-terminal cleavage of a second amino acid useful to generate standards for the measurement of quantities of peptides of interest in a sample.

### CROSS-REFERENCE TO RELATED APPLICATIONS

This application claims the benefit of U.S. Provisional Application No. 62/686,385 filed Jun. 18, 2018. The entirety of this application is hereby incorporated by reference for all purposes.

### STATEMENT REGARDING FEDERALLY SPONSORED RESEARCH OR DEVELOPMENT

This invention was made with government support under AG046161 and AG025688 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 18031PCT_ST25.txt. The text file is 18 KB, was created on Jun. 18, 2019, and is being submitted electronically via EFSWeb.

### BACKGROUND

Mass spectrometry-based proteomic approaches utilizing stable isotope labeled protein or peptide internal standards may be employed for the direct detection and quantification of protein biomarkers. Stable isotope labeling with amino acids in cell culture (SILAC) was developed as a tool for assaying relative concentrations of proteins of cells grown in culture. SILAC incorporates a label into proteins for mass spectrometric (MS)-based proteomics. SILAC relies on metabolic incorporation of a “light” or “heavy” form of an amino acid into proteins. Mass spectrometry using isotopic “heavy” labeled peptide standards are reported to quantify proteins. U.S. Pat. No. 7,939,331 reports mass spectrometry methods of quantifying a biomolecule in a sample that utilize isotopically-labeled biomolecule standards. However, often a large number of analytical replicates are needed for high accuracy for certain biomarker measurements. Thus, there is a need to identify improved methods.

Saveliev et al. report trypsin/Lys-C protease mix for enhanced protein mass spectrometry analysis. Nature Methods, 10, 2013. See also Promega, Discover Reliable Tools for Protein Analysis, Chapter 7, Protein Characterization by Mass Spectrometry. Pages 87-114, 2017, and McDonald et al. Comparison of three directly coupled HPLC MS/MS strategies for identification of proteins from complex mixtures: Single dimension LC-MS/MS, 2-phase MudPIT, and 3-phase MudPIT. Int. J. Mass Spectrom, 219, 245-251 (2002).

Huesgen et al. report LysargiNase mirrors trypsin for protein C-terminal and methylation-site identification. Nature Methods, 12(1): 55-62, 2015.

Wilson reports nispyrtase as a thermostable, N-terminal arginine and lysine specific protease for ≤1 hr digestion, simplified peptide fragmentation and increased MS/MS sensitivity. J Proteomics Bioinform 2014, 7:8.

Kim et al. report detection and quantification of plasma amyloid-β by selected reaction monitoring mass spectrometry. Anal Chim Acta. 2014, 840:1-9.

References cited herein are not an admission of prior art.

### SUMMARY

This disclosure relates to isotopically labeled internal standards that can be digested by an enzyme capable of C-terminal cleavage of a first amino acid and/or an enzyme capable of N-terminal cleavage of a second amino acid useful to generate standards for the measurement of quantities of peptides of interest in a sample. In certain embodiments, the peptide of interest is amyloid precursor protein (APP), Aβ isoforms, Tau, fragments, or variants thereof.

In certain embodiments, this disclosure relates to a parallel enzymatic digestion scheme with mass spectrometry for the detection and quantification of protein biomarkers in tissue and biofluids. In certain embodiments, an object of this disclosure provides: i) detecting protein biomarkers by peptide based comparisons rather than antibody based detection (i.e., immunoassays), ii) reducing assay variability and coefficients of variations, iii) incorporating flanking amino acid residues to facilitate proteolytic digestion and accurate peptide quantification, iv) multiplexing an assay to measure a panel of makers simultaneously and v.) a parallel digesting paradigm utilizing two separate enzymes (lysarginase and nispyrtase, Tryp-C and Tryp-N™) to generate dually tryptic and complementary peptides that in some instances have identical masses, yet have distinguishable fragmentation profiles. One object of the disclosure is to provide robust internal validation that dramatically increases sample throughput by reducing the numbers of technical replicates needed.

In certain embodiments, proteolytic digestion of Tau, a biomarker in Alzheimer's Disease (AD), utilizes two distinct enzymes (lysarginase and nispyrtase, Tryp-C and Tryp-N™), which generate complementary peptide fragments that can be directly detected and quantified by targeted mass spectrometry. By using this complementary tryptic digestion (CompTryp) approach one can measure two overlapping Tau tryptic peptides in the brain. Peptides were equally effective at discriminating patients with AD from controls without AD.

In certain embodiments, this disclosure relates to the design of synthetic isotopically labeled internal standards that can be digested by either Tryp-C and Tryp-N™ to generate complementary standards for the measurement of absolute levels of Tau and beta-amyloid (Aβ) in tissues and biofluids (i.e., plasma and cerebrospinal fluid).

In certain embodiments, methods are applicable to generating peptide surrogates for any disease-related (cancer, chronic inflammation, etc.) protein biomarker from various types of bio-specimens. In certain embodiments, this disclosure contemplates compositions comprising isotope labeled CompTryp peptide standards as disclosed herein and methods of using the compositions to implement mass spectrometry assays for their quantification as protein biomarkers for the early diagnosis of Alzheimer's disease and other chronic disorders.

In certain embodiments, this disclosure relates to methods comprising: providing a sample having a polypeptide of interest to be quantified; providing a copy of the polypeptide of interest, the copy made using at least two amino acids with isotopes having a mass different than that of naturally-occurring isotopes of the polypeptide of interest, wherein the amino acids are two lysine, or two arginine, or a lysine and arginine. In certain embodiments, the method further comprises quantifying the copy polypeptide of interest.

In certain embodiments, the amino acids are two lysine, or two arginine, or a lysine and arginine.

In certain embodiments, the disclosure contemplates that the copy peptide is less than or not longer than 35 amino acids. In certain embodiments, the disclosure contemplates that the copy peptide is less than or not longer than 30 amino acids. In certain embodiments, the disclosure contemplates that the copy peptide is less than or not longer than 25 amino acids.

In certain embodiments, the disclosure contemplates that the copy peptide containing at least a first N-terminal amino acid with an isotope having a mass different than that of the naturally-occurring isotope which is lysine or arginine and a second C-terminal amino acid with isotope having a mass different than that of the naturally-occurring isotope which is not lysine or arginine. Typically, such a first copy peptide would be used in combination with a second copy peptide of the same peptide of interest wherein the second copy peptide comprises two lysines, or two arginines, or a lysine and arginine having a mass different than that of a naturally-occurring isotope which is not lysine or arginine.

In certain embodiments, the method further comprises separating the sample into a first sample and a second sample.

In certain embodiments, the method further comprises introducing a known quantity of the copy into the first sample and the second sample; wherein the copy includes an N-terminal polypeptide linked to a first lysine or a first arginine comprising a first isotope following by a center polypeptide linked to a second lysing or second arginine comprising a second isotope followed by a C-terminal polypeptide.

In certain embodiments, the method further comprises introducing an enzyme capable of N-terminal cleavage of lysine and/or arginine into the first sample, providing an N-terminally cleaved peptide with an N-terminal isotopic lysing or arginine.

In certain embodiments, the method further comprises introducing an enzyme capable of C-terminal cleavage of lysine or arginine into the second sample, providing a C-terminally cleaved peptide with a C-terminal isotopic lysing and/or arginine; analyzing the first sample and second sample by mass spectrometry.

In certain embodiments, the method further comprises comparing obtained mass spectrometry peak pairs resulting from the N-terminally cleaved peptide of the peptide of interest in the first sample, and N-terminally cleaved peptide of the copy to determine a quantity of the peptide of interest in the sample, providing a first quantity of the peptide of interest, wherein the mass spectrometry peak pairs differ in mass by an amount corresponding to the isotope with the different mass taking into account the number of isotope atoms in each ion monitored.

In certain embodiments, the method further comprises comparing obtained mass spectrometry peak pairs resulting from the C-terminally cleaved peptide of the peptide of interest in the second sample, and C-terminally cleaved peptide to determine a quantity of the peptide of interest in the sample, providing a second quantity of the peptide of interest, wherein the mass spectrometry peak pairs differ in mass by an amount corresponding to the isotope with the different mass taking into account the number of isotope atoms in each ion monitored.

In certain embodiments, the method further comprises comparing obtained mass spectrometry peak pairs resulting from the N-terminally cleaved peptide of the peptide of interest in the first sample, and N-terminally cleaved peptide of the copy to determine a quantity of the peptide of interest in the sample, providing a first quantity of the peptide of interest and comparing obtained mass spectrometry peak pairs resulting from the C-terminally cleaved peptide of the peptide of interest in the second sample, and C-terminally cleaved peptide to determine a quantity of the peptide of interest in the sample, providing a second quantity of the peptide of interest wherein the mass spectrometry peak pairs differ in mass by an amount corresponding to the isotope with the different mass taking into account the number of isotope atoms in each ion monitored.

In certain embodiments, the sample is brain tissue, blood, plasma, or cerebrospinal fluid.

In certain embodiments, the copy polypeptide comprises SEQ ID NO: 1 (RSGYSSPGSPGTPGSR). In certain embodiments, the N-terminal arginine (R) comprises the N-terminal isotope and the C-terminal arginine (R) comprises the C-terminal isotope, such as a copy peptide consisting of SEQ ID NO: 2 (SGDR¹SGYSSPGSPGTPGSR²SRT) wherein R¹ and R² contain heavy isotopes.

In certain embodiments, the copy comprises SEQ ID NO: 3 (KTHPHFVIPYR). In certain embodiments, the N-terminal lysine (K) comprises the N-terminal isotope and the C-terminal arginine (R) comprises the C-terminal isotope, such as a copy peptide consisting of SEQ ID NO: 4 (QAK¹THPHFVIPYR²ALV) wherein K¹ and R² contain heavy isotopes.

In certain embodiments, the copy polypeptide comprises SEQ ID NO: 5 (KYLETPGDENEHAHFQK). In certain embodiments, the N-terminal lysine (K) comprises the N-terminal isotope and the C-terminal lysine (K) comprises the C-terminal isotope, such as a copy peptide consisting of SEQ ID NO:6 (AVDK¹YLETPGDENEHAHFQK²AKE) wherein K¹ and K² contain heavy isotopes.

In certain embodiments, the copy polypeptide comprises SEQ ID NO: 7 (RHDSGYEVHHQK). In certain embodiments, the N-terminal arginine (R) comprises the N-terminal isotope and the C-terminal lysine (K) comprises the C-terminal isotope, such as a copy peptide consisting of SEQ ID NO: 8 (AEFR¹HDSGYEVHHQK²LVF) wherein R¹ and K² contain heavy isotopes.

In certain embodiments, the copy polypeptide comprises SEQ ID NO: 9 (KLVFFAEDVGSNK). In certain embodiments, the N-terminal lysine (K) comprises the N-terminal isotope and the C-terminal lysine (K) comprises the C-terminal isotope, such as a copy peptide consisting of SEQ ID NO: 10 (HHQK¹LVFFAEDVGSNK²GAI) wherein K¹ and K² contain heavy isotopes.

In certain embodiments, the copy polypeptide comprises SEQ ID NO: 31 (KGAIIGLMV). In certain embodiments, the N-terminal lysine (K) comprises the N-terminal isotope and the C-terminal valine (V) comprises the C-terminal isotope, such as a copy peptide consisting of SEQ ID NO: 17 (GSNK¹GAIIGLMV²GGVVIAT), SEQ ID NO: 18 (GSNK¹GAIIGLMV²GGVVIA) SEQ ID NO: 19 (GSNK¹GAIIGLMV²GGVV), or SEQ ID NO: 20 (GSNK¹GAIIGLMV²GG) wherein K¹ and V² contain heavy isotopes.

In certain embodiments, the copy polypeptide comprises SEQ ID NO: 32 (KAYQGVAAPFPK). In certain embodiments, the N-terminal lysine (K) comprises the N-terminal isotope and the C-terminal lysine (K) comprises the C-terminal isotope, such as a copy peptide consisting of SEQ ID NO: 21 (PLSK¹AYQGVAAPFPK²ARR) wherein K¹ and K² contain heavy isotopes.

In certain embodiments, the copy polypeptide comprises SEQ ID NO: 33 (RNSEPQDEGELFQGVDPR). In certain embodiments, the N-terminal arginine (R) comprises the N-terminal isotope and the C-terminal arginine (R) comprises the C-terminal isotope, such as a copy peptide consisting of SEQ ID NO: 22 (RGAR¹NSEPQDEGELFQGVDPR²ALA) wherein R¹ and R² contain heavy isotopes.

In certain embodiments, the copy polypeptide comprises SEQ ID NO: 34 (RNSEPQDEGELFQGVDPR). In certain embodiments, the N-terminal arginine (R) comprises the N-terminal isotope and the C-terminal arginine (R) comprises the C-terminal isotope, such as a copy peptide consisting of SEQ ID NO: 22 (RGAR¹NSEPQDEGELFQGVDPR²ALA) wherein R¹ and R² contain heavy isotopes.

In certain embodiments, the copy polypeptide comprises SEQ ID NO: 35 (KAIPVAQDLNAPSDWDSR). In certain embodiments, the N-terminal lysine (K) comprises the N-terminal isotope and the C-terminal arginine (R) comprises the C-terminal isotope, such as a copy peptide consisting of SEQ ID NO: 23 (GAYK¹AIPVAQDLNAPSDWDSR²GKD) wherein K¹ and R² contain heavy isotopes.

In certain embodiments, the copy polypeptide comprises SEQ ID NO: 36 (RGDSVVYGLR). In certain embodiments, the N-terminal arginine (R) comprises the N-terminal isotope and the C-terminal arginine (R) comprises the C-terminal isotope, such as a copy peptide consisting of SEQ ID NO: 24 (YDGR¹GDSVVYGLR²SKS) wherein R¹ and R² contain heavy isotopes.

In certain embodiments, the copy polypeptide comprises SEQ ID NO: 37 (RSYESMCEYQR). In certain embodiments, the N-terminal arginine (R) comprises the N-terminal isotope and the C-terminal arginine (R) comprises the C-terminal isotope, such as a copy peptide consisting of SEQ ID NO: 25 (SDGR¹SYESMCEYQR²AKC) wherein R¹ and R² contain heavy isotopes.

In certain embodiments, the copy polypeptide comprises SEQ ID NO: 38 (RCPCSAVTSTGSCSIK). In certain embodiments, the N-terminal arginine (R) comprises the N-terminal isotope and the C-terminal lysine (K) comprises the C-terminal isotope, such as a copy peptide consisting of SEQ ID NO: 26 (ECLR¹CPCSAVTSTGSCSIK²SSE) wherein R¹ and K² contain heavy isotopes.

In certain embodiments, the copy polypeptide comprises SEQ ID NO: 39 (RSASCDALTGACLNCQENSK). In certain embodiments, the N-terminal arginine (R) comprises the N-terminal isotope and the C-terminal lysine (K) comprises the C-terminal isotope, such as a copy peptide consisting of SEQ ID NO: 27 (CONR¹SASCDALTGACLNCQENSK²GNH) wherein R¹ and K² contain heavy isotopes.

In certain embodiments, the copy polypeptide comprises SEQ ID NO: 40 (KSHEAEVLK). In certain embodiments, the N-terminal lysine (K) comprises the N-terminal isotope and the C-terminal lysine (K) comprises the C-terminal isotope, such as a copy peptide consisting of SEQ ID NO: 28 (ERRK¹SHEAEVLK²QLAE) wherein K¹ and K² contain heavy isotopes.

In certain embodiments, the copy polypeptide comprises SEQ ID NO: 41 (KGIVDQSQQAYQEAFEISKK). In certain embodiments, the N-terminal lysine (K) comprises the N-terminal isotope and the C-terminal lysine (K) comprises the C-terminal isotope, such as a copy peptide consisting of SEQ ID NO: 29 (DDKK¹GIVDQSQQAYQEAFEISK²KEM) wherein K¹ and K² contain heavy isotopes.

In certain embodiments, the copy polypeptide comprises SEQ ID NO: 42 (KSVTEQGAELSNEER). In certain embodiments, the N-terminal lysine (K) comprises the N-terminal isotope and the C-terminal arginine (R) comprises the C-terminal isotope, such as a copy peptide consisting of SEQ ID NO: 30 (ACMK¹SVTEQGAELSNEER²NLL) wherein K¹ and R² contain heavy isotopes.

In certain embodiments, the first quantity, second quantity, or combination thereof, of the peptide of interest, is used to diagnosed or monitor Alzheimer's disease (AD), corticobasal degeneration (CBD), frontotemporal dementia (FTD), or other neurodegenerative diseases or tauopathies.

In certain embodiments, this disclosure relates to a polypeptide comprising an N-terminal polypeptide linked to a first lysine or a first arginine comprising a first isotope following by center polypeptide linked to a second lysing or second arginine comprising a second isotope followed by a C-terminal polypeptide. In certain embodiments, the N-terminal polypeptide is a tripepetide or dipeptide. In certain embodiments, the C-terminal polypeptide is a tripepetide or dipeptide.

In certain embodiments the peptide comprises or consists of SEQ ID NO: 1 (RSGYSSPGSPGTPGSR), SEQ ID NO: 3 (KTHPHFVIPYR), SEQ ID NO: 5 (KYLETPGDENEHAHFQK), SEQ ID NO: 7 (RHDSGYEVHHQK), SEQ ID NO: 9 (KLVFFAEDVGSNK), SEQ ID NO: 31 (KGAIIGLMV), SEQ ID NO: 32 (KAYQGVAAPFPK), SEQ ID NO: 33 (RNSEPQDEGELFQGVDPR), SEQ ID NO: 34 (RNSEPQDEGELFQGVDPR), SEQ ID NO: 35 (KAIPVAQDLNAPSDWDSR), SEQ ID NO: 36 (RGDSVVYGLR), SEQ ID NO: 37 (RSYESMCEYQR), SEQ ID NO: 38 (RCPCSAVTSTGSCSIK), SEQ ID NO: 39 (RSASCDALTGACLNCQENSK), SEQ ID NO: 40 (KSHEAEVLK), SEQ ID NO: 41 (KGIVDQSQQAYQEAFEISKK), SEQ ID NO: 42 (KSVTEQGAELSNEER) or combinations thereof.

In certain embodiments the peptide comprises or consists of SEQ ID NO: 2 (SGDRSGYSSPGSPGTPGSRSRT), SEQ ID NO: 4 (QAKTHPHFVIPYRALV), SEQ ID NO: 6 (AVDKYLETPGDENEHAHFQKAKE), SEQ ID NO: 8 (AEFRHDSGYEVHHQKLVF), SEQ ID NO: 10 (HHQKLVFFAEDVGSNKGAI), SEQ ID NO: 17 (GSN KGAIIG LMVGGVVIAT), SEQ ID NO: 18 (GSNKGAIIGLMVGGVVIA), SEQ ID NO: 19 (GSNKGAIIGLMVGGVV), SEQ ID NO: 20 (GSN KGAIIGLMVGG), SEQ ID NO: 21 (PLSKAYQGVAAPFPKARR), SEQ ID NO: 22 (RGARNSEPQDEGELFQGVDPRALA), SEQ ID NO: 23 (GAYKAIPVAQDLNAPSDWDSRGKD), SEQ ID NO: 24 (YDGRGDSVVYGLRSKS), SEQ ID NO: 25 (SDGRSYESMCEYQRAKC), SEQ ID NO: 26 (ECLRCPCSAVTSTGSCSIKSSE), SEQ ID NO: 27 (CNNRSASCDALTGACLNCQENSKGNH), SEQ ID NO: 28 (ERRKSHEAEVLKQLAE), SEQ ID NO: 29 (DDKKGIVDQSQQAYQEAFEISKKEM), SEQ ID NO: 30 (ACMKSVTEQGAELSNEERNLL), or combinations thereof.

In certain embodiments, this disclosure relates to kits comprising, 1) the copy of the polypeptide of interest as disclosed herein, 2) an enzyme capable of N-terminal cleave of lysine and arginine, and/or 3) an enzyme capable of C-terminal cleave of lysine or arginine.

### BRIEF DESCRIPTION OF THE DRAWINGS

FIG. 1. Absolute quantification of Aβ peptide in human AD brain tissue. A fully tryptic peptide amyloid β (Aβ) specific peptide (position 17-28) (SEQ ID NO: 11); was isotopically labeled at lysine (K, asterisks) and spiked into a pool of AD brain peptides at a known concentration (1 μM). The complexity of peptides in the sample is highlighted by the base peak chromatogram. By exploiting the high resolution and accurate mass capabilities of the Orbitrap mass spectrometer both the light (native) and heavy (standard) can be readily resolved by LC-MS/MS. Notably the heavy and light peptide co-elute, yet can be distinguished by the mass shift introduced by heavy stable isotopes (e.g. 6C¹³ and 2N¹⁵) on lysine within the standard. The ratio of the light over the heavy allows for absolute quantification of Aβ in brain extracts.

FIG. 2 shows a complementary trypsin digestion scheme to measure absolute Tau levels in AD brain. A CompTryp standard peptide with two heavy labeled arginine residues for Tau (residues 194-209) adjacent to three flanking residues (underlined) (SEQ ID NO: 2) to control for protein digestion. After spiking the CompTryp Tau peptide at a known concentration (20 fmol) into brain homogenates two independent digestions occur. When the Tau ComTryp standard is spiked a known concentration into human brain tissue and digested with TrypC, the C-terminal tryptic peptide will be predominately generated SGYSSPGSPGTPGSR (SEQ ID NO: 12), whereas a separate digestion with Tryp-N™ will generate the N-terminal tryptic peptide RSGYSSPGSPGTPGS (SEQ ID NO: 13). Both the endogenous light Tau peptides (arginine) and heavy standards (arginine) Tau standards can be resolved and quantified in the parallel reaction monitoring (PRM) in the mass spectrometer.

FIG. 3 illustrates digested peptide (SEQ ID NO: 13) and undigested (SEQ ID NO: 2) or partially digested intermediates (SEQ ID NOs: 14 and 15) after 8 h. A CompTryp standard for tau peptide SGDRSGYSSPGSPGTPGSRSRT (SEQ ID NO: 2) is flanked by amino acid residues that essentially mimic the protein sequence of full-length tau. Theoretically the mass spectrometer can detect both the fully trypsinC or TrypN cleaved peptide (#6) and any residual or undigested products (1-5), which have different elution profiles, to ensure complete digestion. Amino acids are isotopically labeled. TrypC and TrypN were 99.8% and 94.1% efficient, respectively, in digesting this Tau CompTryp Standard.

FIG. 4 illustrates heavy labeled synthetic Tau Tryp-N™ and Tryp-C peptides co-elute with Tau in AD brain. The Tryp-N™ and Tryp-C Tau light peptides from AD brain have the exact same m/z detected (697.3223), yet co-elute 30 seconds apart. This Tryp-N™ elutes at 14.22 min and Tryp-C at 14.92 mins. The isotopic standards co-elute with the light peptides, yet at are 10 Da or 5 m/z heavier (702.3257).

FIG. 5A shows data indicating reproducible measurements of Tau levels in control and AD human brain tissue after Tryp-C and Tryp-N™ digestion. This ratio of endogenous light Tau peptide compared to the heavy standard following Tryp-C digestion or Tryp-N™. Relative endogenous Tau levels determined by Tryp-N™ (y-axis) or Tryp-C(x-axis) were highly correlated.

FIG. 5B shows data indicating Tau levels by PRM correlate (R2=0.9553) well with their respective total Tau ELISA measures.

FIG. 6A illustrates A4_HUMAN Isoform APP 695 of Amyloid beta A4 protein P05067-4. The Aβ sequence is underlined.

FIG. 6B shows sequence coverage by synthetic peptide standards: Tryp-C and Tryp-N™ digestion to measure APP and Aβ isoforms, specific to the N-terminal of APP, common to APP and those that are Aβ isoform specific. The residues are the proposed heavy isotopic (³C, ¹⁵N) versions of the peptides. The peptide A1316-28 is flanked by amino acid residues (underlined) that mimic the protein sequence of full-length amyloid precursor protein (APP).

FIG. 7 shows data indicating the Tryp-C and Tryp-N™ digestion improve accuracy of Tau measurement in human CSF. The percent variation for TrypN™ and TrypC measurements alone or averaged. The variance is significantly reduced when you average the measurements (* p<0.05).

FIG. 8 CompTryp standards measure Aβ 40 and 42 isoforms in human CSF. The ratio of Aβ42/40 was calculated from PRM measurements of CompTryp peptides for Aβ28-40 and Aβ28-42 (See FIG. 6B).

FIG. 9A shows data in the brain indicating SMOC1 is significantly increased in both AD (n=9) and AsymAD (n=8) compared controls (n=10).

FIG. 9B shows data indication SMOC1 levels are also significantly higher in AD (n=40) compared to non-AD controls (n=40) using MS and heavy labeled CompTryp standards. For SMOC1 MS assays global pooled standards (std.) were analyzed 10 times to assess technical variance.

FIG. 9C shows data indicating aggregated level of multiple brain derived proteins quantified by MS in CSF from the inflammation module (n=28) discriminates AD from non-AD controls (n=20 samples each group).

FIG. 10 shows a table of copy peptides that have been synthesized can be used as peripheral CSF biomarker panel designed specifically to measure the pathophysiologies that underlie AD in human brain.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim. In certain embodiments, the disclosure contemplates that the “N-terminus of a peptide may consist of an amino acid sequence,” which refers to the N-terminus of the peptide having the exact number of amino acids in the sequence and not more or having not more than a rage of amino acids specified in the claim however the C-terminus may be connected to additional amino acids, e.g., as part of a larger peptide. Similarly, the disclosure contemplates that the “C-terminus of a peptide may consist of an amino acid sequence,” which refers to the C-terminus of the peptide having the exact number of amino acids in the sequence and not more or having not more than a rage of amino acids specified in the claim however the N-terminus may be connected to additional amino acids, e.g., as part of a larger peptide.

As used herein the terms “amino acids with isotope having a mass different than that of naturally-occurring isotopes” refers to elemental(s) in the amino acid which have an isotope distribution that is altered compared to those found in nature (observed natural abundance of each element). Atoms that have the same number of protons but different numbers of neutrons are known as isotopes. Natural abundances of all the stable isotopes varies for each element. Often a heavy isotope is enriched in an element resulting in an amino acid that has a heavier mass than that of the naturally-occurring isotope.

This disclosure relates to isotopically labeled heavy internal standards that can be digested by an enzyme capable of C-terminal cleavage of a first amino acid and/or an enzyme capable of N-terminal cleavage of a second amino acid useful to generate standards for the measurement of quantities of peptides of interest in a sample.

Amyloid precursor protein (APP) fragments (amyloid-β (Aβ) peptides) and Tau are the main components of senile plaques. Aβ peptides vary in length due to processing of β- and γ-secretases on amyloid precursor protein (APP). Protein fragments are normally degraded and eliminated. However, the 42-amino acid peptide (Aβ42) is prone to form aggregated amyloid oligomers in patients diagnosed with dementia.

Tau is a neuronal protein. Six isoforms of microtubule-associated protein tau (MAPT) are expressed in the adult human brain. Tau proteins have phosphorylation sites and phosphorylation patterns alter the ability of Tau to bind and promote microtubule assembly. Hyper phosphorylation of Tau, intracellular Tau deposits, abnormal Tau splicing, and Tau gene mutations are reported to be associated with neurodegenerative conditions and diseases such as frontotemporal dementia, Alzheimer's disease (AD), Pick disease, and progressive supranuclear palsy (PSP).

Disclosed herein is paradigm for the generation of heavy labeled peptide standards that provides robust internal validation, and dramatically increases sample throughput by reducing the numbers of technical replicates needed. Experiments have been performed to evaluate proteolytic digestion of Tau, a biomarker for AD, using two distinct enzymes (Tryp-C and Tryp-N™) which generate complementary peptide fragments for the direct detection and quantification by mass spectrometry. Notably, Tau levels determined by both independent tryptic peptides were highly correlated and equally effective at discriminating patients with AD from controls in human brain tissue. Thus, engineered isotopically labeled heavy internal standards can be digested by either Tryp-C and Tryp-N™ to generate complementary standards for the measurement of relative and absolute levels protein biomarkers in clinical samples. Moreover, these methods are broadly applicable to generating peptide surrogates for any disease-related (cancer, chronic inflammation, etc.) protein biomarker.

In certain embodiments, this disclosure relates to the design of synthetic isotopically labeled heavy internal standards that can be digested by TrypC and Tryp-N™ to generate complementary standards for the measurement of absolute levels of Tau and beta-amyloid (Aβ) in tissues and biofluids (i.e., plasma and cerebrospinal fluid).

In certain embodiments, methods are applicable to generating peptide surrogates for any disease-related (cancer, chronic inflammation, etc.) protein biomarker from various types of bio-specimens. In certain embodiments, this disclosure contemplates compositions comprising isotope labeled CompTryp peptide standards as disclosed herein and methods of using the compositions to implement mass spectrometry assays for their quantification as protein biomarkers for the early diagnosis of Alzheimer's disease and other chronic disorders.

In certain embodiments, this disclosure relates to methods comprising: providing a sample having a polypeptide of interest to be quantified; providing a copy of the polypeptide of interest, the copy made using at least two amino acids with isotope having a mass different than that of naturally-occurring isotopes of the polypeptide of interest, wherein the amino acid are two lysine, or two arginine, or a lysine and arginine. In certain embodiments, the method further comprises quantifying the copy polypeptide of interest.

In certain embodiments, the method further comprises separating the sample into a first sample and a second sample.

In certain embodiments, the method further comprises introducing a known quantity of the copy into the first sample and the second sample; wherein the copy includes an N-terminal polypeptide linked to a first lysine or a first arginine comprising a first isotope following by a center polypeptide linked to a second lysing or second arginine comprising a second isotope followed by a C-terminal polypeptide.

In certain embodiments, the method further comprises introducing an enzyme capable of N-terminal cleavage of lysine and arginine into the first sample, providing an N-terminally cleaved peptide with an N-terminal isotopic lysing or arginine.

In certain embodiments, the method further comprises introducing an enzyme cable of C-terminal cleavage of lysine or arginine into the second sample, providing a C-terminally cleaved peptide with a C-terminal isotopic lysing or arginine; analyzing the first sample and second sample by mass spectrometry.

In certain embodiments, the method further comprises comparing obtained mass spectrometry peak pairs resulting from the N-terminally cleaved peptide of the peptide of interest in the first sample, and N-terminally cleaved peptide of the copy to determine a quantity of the peptide of interest in the sample, providing a first quantity of the peptide of interest, wherein the mass spectrometry peak pairs differ in mass by an amount corresponding to the isotope with the different mass taking into account the number of isotope atoms in each ion monitored.

In certain embodiments, the method further comprises comparing obtained mass spectrometry peak pairs resulting from the C-terminally cleaved peptide of the peptide of interest in the second sample, and C-terminally cleaved peptide to determine a quantity of the peptide of interest in the sample, providing a second quantity of the peptide of interest, wherein the mass spectrometry peak pairs differ in mass by an amount corresponding to the isotope with the different mass taking into account the number of isotope atoms in each ion monitored.

In certain embodiments, the method further comprises comparing obtained mass spectrometry peak pairs resulting from t the N-terminally cleaved peptide of the peptide of interest in the first sample, and N-terminally cleaved peptide of the copy to determine a quantity of the peptide of interest in the sample, providing a first quantity of the peptide of interest and comparing obtained mass spectrometry peak pairs resulting from the C-terminally cleaved peptide of the peptide of interest in the second sample, and C-terminally cleaved peptide to determine a quantity of the peptide of interest in the sample, providing a second quantity of the peptide of interest wherein the mass spectrometry peak pairs differ in mass by an amount corresponding to the isotope with the different mass taking into account the number of isotope atoms in each ion monitored.

The present disclosure is advantageous over prior methods for example, in enabling accurate quantitation of biomolecules analyzed by MS by spiking a copy of a biomolecule standard into a sample with Tryp-C and Tryp-N™ resulting in fragment peptide copies. The peptide fragment copy is differentiable under MS from the biomolecule copied, for example by the incorporation therein of an isotopic label. Thus, knowing the quantity and/or concentration of the peptide fragment copy introduced into the MS process allows an accurate determination of the relative or absolute quantity of the biomolecule copied. Alternatively, relative changes could be analyzed if the same amount (e.g., volume) of the copy of a biomolecule standard is added to two samples and relative peak intensities are analyzed. Comparison of concentrations of a peptide of interest in two samples may be enabled, with or without determining an absolute concentration of the copy molecule.

The present disclosure can be used for quantitation of one or more polypeptides in a sample. The methods include adding an isotopically-labeled copy polypeptide of interest of known quantity to a sample with Tryp-C and Tryp-N™ that contains the polypeptide of interest (or a variant thereof, for example, a splice variant, a polypeptide generated from a different member of a gene family, a variant having one or more post-translational modifications); analyzing the sample by mass spectrometry; and comparing the mass spectrometry peaks of pairs resulting from fragments of the isotopically-labeled copy polypeptide of interest and the polypeptide of interest of the sample, in which the fragments of isotopically-labeled copy polypeptide differs from the mass of the polypeptide of interest by an amount corresponding to the difference in mass between the labeling isotope and the cognate naturally-occurring isotope, taking into account the number of labeled isotopes incorporated into the polypeptide. The methods further include comparing peak heights of the members of a peak pair to quantitate the polypeptide of interest in the sample, taking into account the known mass of the added copy polypeptide standard.

In a typical embodiment, the polypeptide copy and the polypeptides of the sample are fragmented with Tryp-C and Tryp-N™ prior to MS. In these embodiments, fragment peaks are compared and used to quantitate the amount of individual fragments of a polypeptide of interest. In some aspects, multiple fragments of a copy polypeptide can thus be used to assess the internal consistency of the quantitation for a given polypeptide of interest. In other aspects, multiple fragments of a copy polypeptide can be used to determine the relative and absolute abundance of splice variants or differentially modified variants of a protein of the sample. In these aspects, for example, at least one fragment generated from the copy polypeptide prior to MS is preferably within or substantially overlaps a protein domain that is encoded by an alternatively spliced exon of a gene encoding the polypeptide of interest, or is within or substantially overlaps a domain of the protein that can be proteolytically processed in a cell, or includes a post-translational modification site.

A “copy” of a biomolecule may be for example, a biomolecule, such as a polypeptide that behaves in a chemical and electrostatic fashion that is essentially identical to the polypeptide copied. The copy may be longer or shorter than the copied polypeptide and/or may differ in sequence in some regions from the copied polypeptide. However, at least one fragment of the copy, for example, a tryptic fragment, will behave ionicly and chemically as a fragment of the same region of the polypeptide copied. By way of non-limiting example, arginines or lysines synthesized with a heavy nitrogen isotope will have essentially the same biological and chemical properties as natural arginine or lysine. When this heavy arginine or lysine is incorporated into a copy polypeptide, the arginine or lysine residue will behave the essentially the same whether it is natural or a copy; the protein will similarly behave the same whether containing a heavy or light isotope. Cleavage at these residues, for example, by a tryptic enzyme, will produce a peptide fragment of heavier mass due to the terminal high mass arginine and/or lysine, but with the same chemical and ionizing characteristics as the peptide fragment from a natural peptide. For example, the tryptic enzyme may be trypsin or an enzyme with cleavage characteristics similar to trypsin. The high mass polypeptide from which the high mass peptide is cleaved is thus for the purposes of the present disclosure considered a copy of the natural polypeptide, although flanking regions of the copy peptide fragment may or may not have the same characteristics as flanking regions of the natural polypeptide. For example, as discussed further below, the copy may include a tag or marker to facilitate or monitor synthesis and/or to aid purification.

As used herein, the terms “heavy-isotope labeled biomolecule,” and “isotopically-labeled biomolecule” mean a biomolecule that has incorporated in its chemical structure, an isotope of an atom that is different from the predominant isotope found in nature. In context, an atom refers to a plurality of atoms having the same atomic number.

In various aspects, the disclosure includes mass spectrometry. As used herein, the term “mass spectrometry” (or simply “MS”) encompasses any spectrometric technique or process in which molecules are ionized and separated and/or analyzed based on their respective molecular weights. Thus, as used herein, the terms “mass spectrometry” and “MS” encompass any type of ionization method, including without limitation electrospray ionization (ESI), atmospheric-pressure chemical ionization (APCI) and other forms of atmospheric pressure ionization (API), and laser irradiation. Mass spectrometers are commonly combined with separation methods such as gas chromatography (GC) and liquid chromatography (LC). GC or LC separates the components in a mixture, and the components are then individually introduced into the mass spectrometer; such techniques are generally called GC/MS and LC/MS, respectively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the disclosure. In such combinations, “MS” can refer to any form of mass spectrometry; by way of non-limiting example, “LC/MS” encompasses LC/ESI MS and LC/MALDI-TOF MS. Thus, as used herein, the terms “mass spectrometry” and “MS” include without limitation APCI MS; ESI MS; GC MS; MALDI-TOF MS; LC/MS combinations; LC/MS/MS combinations; MS/MS combinations; etc.

“Synthesizing” a polypeptide, biomolecule, protein, and the like, simply refers to tools that might be used for peptide synthesis, for example, a gene, host cell, in vitro translation system, a buffer, a medium, amino acids, amino acid precursors, a solid phase synthesis system, or component part thereof.

As used herein, a “polypeptide” is a polymer of amino acids linked by peptide bonds. As used herein, a “protein” is a polypeptide, that is, a polymer of amino acids. The terms “protein” and “polypeptide” are used somewhat interchangeably herein. While the protein may have biologic activity, no inference of a requirement for activity is to be made. A protein may contain modifications to the amino acid monomers. For example, a protein may be glycosylated or phosphorylated. “Protein” includes protein containing molecules such as lipoproteins and glycoproteins. The terms “polypeptide” and “peptide” are used somewhat interchangeably herein. In the art, “peptide” is simply a molecule containing peptide bonds. The term “peptide” is generally used to describe fragments of a protein or polypeptide, for example, resulting from enzymatic or chemical digestion, for example, using a tryptic enzyme or cyanogen bromide. But the term “polypeptide” may similarly include fragments thereof. In analysis by mass spectrometry, a polypeptide is often split into multiple peptides, for example, two, three, four, five, six, seven or more peptide components from the polypeptide. Analysis of any one of these peptide fragments yields information with respect to the source polypeptide. When data from plural peptides are confirmatory, confidence in the analysis is increased. Terms such as “polypeptide of interest”, “protein of interest” and “biomolecule of interest” include any source polypeptide, protein or biomolecule or peptide bond containing fragment thereof about which an investigator seeks information.

A “substantially non-radioactive isotope” is an isotope or mixture of isotopes whose disposal is not regulated by rules designed to protect the public from radiation exposure.

A “synthesized copy” is a copy made by any non-natural means, that is, a copy made as a result of the intervention of man. For example, synthesis may be accomplished by providing chemicals and reaction conditions in sequence to produce a desired biomolecule; by providing an in vitro translation system, such as a cell extract in vitro transcription/translation system; by providing a host cell (prokaryotic or eukaryotic) and a template for the host cell to use for manufacturing a biomolecule of interest such as a polypeptide; or by providing chemicals and/or catalysts, such as enzymes, to produce a desired biomolecule. The synthetic process may include a concentration process, a purification process, an enriching process, etc. including photoreactive processes to facilitate use of the synthesized copy in a desired process or analysis.

The present disclosure includes methods for quantifying one or more biomolecules, such as proteins or other polypeptides in a sample using mass spectrometry. Such methods may include providing at least one sample having a biomolecule of interest to be quantified; providing a copy of the biomolecule of interest, in which the copy is made using an isotope having a mass different than that of naturally-occurring isotopes of the biomolecule of interest; quantifying the biomolecule of interest in the copy; introducing a known quantity of the copy into said sample; cleaving an N-terminal and C-terminal peptide of the copy; analyzing the sample by mass spectrometry; and comparing obtained mass spectrometry peak pairs resulting from the one or more biomolecule or a fragment thereof, and the copy or a fragment thereof to determine the quantity of the mass spectrometry peak pairs differ in mass by an amount corresponding to the isotope with the different mass taking into account the number of isotope atoms in each ion monitored.

Other embodiments of the present disclosure include methods for quantifying one or more biomolecules using a biomolecule standard made using recombinant expression. Such methods may include for example, providing at least one sample having a biomolecule of interest to be quantified; providing a copy of the biomolecule of interest, in which the copy is made using recombinant expression and the copy includes an isotope having a mass different than that of naturally occurring isotopes of the biomolecule of interest; quantifying the biomolecule of interest in the copy; introducing a known quantity of the copy into the sample; cleaving an N-terminal and C-terminal peptide of the copy; analyzing the sample by mass spectrometry; and comparing obtained mass spectrometry peak pairs resulting from the one or more biomolecules or one or more fragments thereof, and the copy or one or more fragments thereof to determine the quantity of the one or more biomolecules in the one or more samples.

Samples in accordance with the present disclosure may include for example, at least one component selected from the group consisting of biological cells, a cell supernatant, a cell extract, embryos, a cell lysate, viruses, biological tissue, a tissue slice, an organ, an organism, a collection of organisms, a portion of an organism, a biopsy, a sample of bodily fluid, a blood sample, a serum sample, and a cell-free biological mimetic system.

The copy of the biomolecule of interest is labeled with at least one isotope, or at least two isotopes, to form a standard. Non-limiting examples of isotopes for labeling include ²H, ¹³C, ¹⁵N, ¹⁷O, ¹⁸O, ³⁴S, etc. Preferably, the isotope is a heavy isotope that is substantially non-radioactive isotope. By isotope-labeled biomolecule, is meant a biomolecule that has incorporated in its chemical structure an isotope of an atom that is different from the predominant isotope found in nature. Thus, the label is a mass-altering label. The mass of the label should be such that upon MS analysis, a peak associated with the biomolecule is distinguishable from a peak associated with the isotope-labeled biomolecule.

The biomolecule standard may be chemically synthesized or may take advantage of biological processes or molecules. The labeled atom or molecule is incorporated into the biomolecule and spiked into a sample for quantitation of one or more biomolecules in the sample. According to certain embodiments, the copy of the biomolecule (e.g., polypeptide or protein) of interest may be made by example, using free cell synthesis or an engineered cell. Engineered cells of the present disclosure may be cells derived from a cell or cell line other than a cell or cell line from which the sample is obtained.

According to certain embodiments, the biomolecule standard may be made using recombinant methods, such as in vitro translation and synthesis in an engineered cell or a cultured cell. For example, copies of the biomolecule may be made by expressing the biomolecule from an engineered construct in a cultured cell (which can be episomal or incorporated into a host cell chromosome), or from a cell that has been engineered (genetically modified) to overexpress or inducibly express an endogenous gene or a gene recombined into the cell's genome (recombinant cells). Generation of the reference standard for quantitative analysis of expressed proteins by recombinant methods has a number of advantages over using a control cell culture. One advantage is the reduced amount and cost of isotopically-labeled amino acid. Another advantage is the ability to efficiently synthesize isotopically-labeled macromolecules. Methods for making biomolecule standards using recombinant methods are described further below.

The copy of the polypeptide includes at least one isotope in an amino acid or amino acid residue of the polypeptide, preferably two isotopes. Non-limiting examples of the amino acids or amino acid residues useful for making isotopically-labeled polypeptide copies include those selected from the group consisting of Arg, Lys, Asp, Glu, Met, Trp, Ser, Thr, Tyr, Val, and Asn.

According to certain embodiments, the copy may be made using an isotope pool. For example, the labeling isotope may be present in a pool of molecules containing the labeling isotope as a predominant fraction of the pool. The isotope pool may include for example, substantially non-radioactive isotopes. By way of non-limiting example, the isotope pool may include at least one isotope having a mass distribution different than that of naturally occurring isotopes in the polypeptide of interest. For example, the labeling isotope may be present in a pool of molecules, such as a pool of amino acids including one or more of, for example, arginine, threonine, serine, tyrosine molecules in a fraction for example at least about 100%, 99%, 98%, 95%, 90%, 80%, 75% of the pool. Higher fractions are expected to provide higher quality data.

Certain embodiments for example, use amino acids labeled with ¹⁵N or ¹³C, more preferably ¹⁵N-Arg or ¹³C-Arg, or ¹⁵N-lys or ¹³C-lys. This expression will be used as an exemplary shorthand expression for the heavy-isotope labeled biomolecules of the present disclosure. For example, a pool of ¹⁵N-Arg would include a significant, i.e., measurable, difference from a natural pool of Arg. The pool of atoms, e.g. a pool where ¹⁵N predominates may be incorporated into a biomolecule. Labeled biomolecules synthesized using the pool will be distinguishable, for example in MS from natural forms of the biomolecule.

A biomolecule standard may be quantified, for example, by biochemical or spectroscopic methods as known in the art for the particular type of biomolecule. The biomolecule standard may then be used for example, as a quantitative internal reference for a sample cell lysate. A protein standard can be quantified by protein quantitation assays as they are known in the art, for example, ELISA, Bradford assays, Lowry assays, bicinchoninic acid (BCA) assays, or modified versions of these, or fluorescence based assays, such as are commercially available.

The biomolecule standard may then be introduced into the sample to be analyzed. According to certain embodiments, the quantitation standard is introduced into the sample as early in the processing of the sample as possible. This ensures that losses or modifications induced by the fractionation process occur evenly across the sample components and the reference standard(s).

Mass spectrometry provides a rapid and sensitive technique for the characterization of a wide variety of molecules. In the analysis of peptides and proteins, mass spectrometry can provide detailed information regarding, for example, the molecular mass (also referred to as “molecular weight” or “MW”) of the original molecule, the molecular masses of peptides generated by proteolytic digestion of the original molecule, the molecular masses of fragments generated during the ionization of the original molecule, and even peptide sequence information for the original molecule and fragments thereof.

A time-of-flight mass spectrometer determines the molecular mass of chemical compounds by separating the corresponding molecular ions according to their mass-to-charge ratio (the “m/z value”). Ions are accelerated in the presence of an electrical field, and the time necessary for each ionic species to reach a detector is determined by the spectrometer. The “time-of-flight” values obtained from such determinations are inversely proportional to the square root of the m/z value of the ion. Molecular masses are subsequently determined using the m/z values once the nature of the charged species has been elucidated.

A particular type of MS technique, matrix-assisted laser desorption time-of-flight mass spectrometry (MALDI-TOF MS) (Karas et al., Int. J. Mass Spectrom. Ion Processes 78:53, 1987), has received prominence in analysis of biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Nevertheless, matrix molecules transfer their energy to analyte molecules, causing them to vaporize and ionize. The ionized molecules are accelerated in an electric field and enter the flight tube. During their flight in this tube, different molecules are separated according to their mass to charge (m/z) ratio and reach the detector at different times. Each molecule yields a distinct signal. The method is used for detection and characterization of biomolecules, such as proteins, peptides, oligosaccharides and oligonucleotides, with molecular masses between about 400 and about 500,000 Da, or higher. MALDI-MS is a sensitive technique that allows the detection of low (10¹⁵ to 10¹⁸ mole) quantities of analyte in a sample.

Partial amino acid sequences of proteins can be determined by enzymatic or chemical proteolysis followed by MS analysis of the product peptides. These amino acid sequences can be used for in silico examination of DNA and/or protein sequence databases. Matched amino acid sequences can indicate proteins, domains and/or motifs having a known function and/or tertiary structure. For example, amino acid sequences from an uncharacterized protein might match the sequence or structure of a domain or motif that binds a ligand. As another example, the amino acid sequences identified by MS analysis can be used as antigens to generate antibodies to the protein and other related proteins from other biological source material (e.g., from a different tissue or organ, or from another species). There are many additional uses for MS, particularly MALDI-TOF MS, in the fields of genomics, proteomics and drug discovery. For a general review of the use of MALDI-TOF MS in proteomics and genomics, see Bonk et al. (Neuroscientist 7:12, 2001).

Peptides labeled with light or heavy amino acids can be directly analyzed using MALDI-TOF. However, where sample complexity is apparent, on-line or off-line LC-MS/MS or two-dimensional LC-MS/MS may be necessary to separate the peptides.

MS may be capable of resolving the heavy and light isotope isoforms for each peptide, as well as measuring the relative intensity of each isoform. After MS is performed, mass spectrometry peak pairs result from each biomolecule being analyzed and its corresponding biomolecule standard having a different molecular weight. The mass spectrometry peak pairs may be compared to determine either an absolute or relative quantity of the one or more biomolecules in the sample. According to certain embodiments, the present disclosure features use of the protein standard to quantify proteins that share regions of sequence identity (such as homologous proteins or splice variants).

Methods of the present disclosure may further include forming further fragments of the biomolecule of interest, for example, by digestion with an enzyme or cleavage with a chemical reagent. Enzymes in accordance with the present disclosure may include for example, at least one enzyme selected from the group consisting of a protease (for example, a serine protease), a phosphorylase, a peptidase, a diesterase, a lipase and an oxidoreductase, a transferase, a hydrolase, a lyase, an isomerase, and a ligase.

According to certain embodiments, the at least one sample includes a plurality of samples. At least a first sample of the plurality of samples may have been exposed to a different set of conditions than a second sample of the plurality of samples. Non-limiting examples of different sets of conditions may include for example, species or strain from which the sample is obtained, disease-state versus normal state of an organism from which the sample is obtained; one or more of: chemical exposure; genetic manipulation; feeding regimen; environmental difference; aging or developmental stage; or difference in exposure to one or more hormones, growth factors, or cytokines.

Methods according to the present disclosure may include providing at least a second sample whose polypeptide of interest is desired to be quantified; introducing a known quantity of the copy into the second sample; cleaving an N-terminal and C-terminal peptide of the copy; analyzing by mass spectrometry the second sample to determine a quantity of polypeptides of interest or a fragment thereof in said second sample; and comparing the quantity of at least one polypeptides of interest or one or more fragments thereof of the sample to the second sample. According to certain embodiments, the second sample may be from a preparation different from the sample.

According to certain embodiments, biomolecules of interest, such as proteins of interest, may be quantified from a cell lysate. Cells may be grown in culture or obtained from a tissue sample from an organism, for example, the tissue sample may be a tissue slice or a biopsy, for example a needle biopsy. The sample may be obtained for example, from a blood sample. The cell lysate is prepared from a cell population. Lysis can be accomplished in any fashion, for example, by detergent disruption, sonication, osmotic shock, freezing, etc. Embodiments of the disclosure may include for example, quantifying proteins from a cell lysate that originated from cells that expressed proteins recombinantly or naturally in cell culture, as well as samples that originated from plasma, serum, CSF, urine, sputum, semen, lymph, or any biological fluid, tissue or other sample. According to other embodiments of the present disclosure, proteins may be quantified from a cell lysate that originated from cells that have been challenged by an external stimulus such as application of a drug, radiation, or a change in the environment, such as a feeding schedule, temperature change, light dark cycle, etc.

According to other embodiments of the present disclosure, biomolecules, such as proteins may be quantified from healthy samples and compared to proteins quantified from disease samples. Comparing protein expression patterns between normal and disease conditions may reveal proteins whose changes are important in the disease aid thus, identify proteins which aid diagnosis or whose modification may be of therapeutic value. In clinical research, changes in protein expression may be useful indicators of targets of treatments, efficacy of treatments or monitoring unknown side effects of treatments. In basic research, changes in protein expression may be useful indicators of responses of a cell to experimental manipulation. Methods of detecting protein expression profiles may also have important applications in, for example and without limitation, tissue typing, drug screening, forensic identification, and clinical diagnosis.

Certain embodiments of the disclosure are directed to drug or toxicology screening. For example, a sample from a culture, a tissue or an organism exposed to a drug, a drug metabolite, a drug candidate or a drug candidate metabolite, may be analyzed to test efficacy, toxic effect, alternate uses, etc.

Another aspect of the present disclosure may allow monitoring effects of stimuli or conditions, such as feed regimens, growth factors, cytokines, temperatures, pH, etc., for effects such as phenotypic effects on a cell, organism, or tissue. For example, differentiation or dedifferentiation, for example of adult or stem cells, e.g., differentiated cells or stem cells, hematopoietic stem cells, neural stem cells, cardiac stem cells, etc., may be monitored for differentiation or dedifferentiation markers. Cells in this aspect as well as other aspects may be human or other animal, for example, mammal, insect or fish. Rodent cells such as rat or mouse may be used. Food producing organisms such as plant or meat producing animals are likewise suitable sources for sample material and/or cells. Corn, grain, wheat, rice, ovine, porcine, equine, etc. tissues may supply samples. Samples may be used for possible commercial development or for experimental convenience.

Certain embodiments of the present disclosure include using at least one manufactured protein, for example, a recombinantly expressed and heavy-isotope labeled reference protein and/or set of proteins. A single protein may be investigated. However, multiple proteins or polypeptides can be manufactured to contain a heavy isotope. Any or all of the manufactured proteins can be spiked into a sample. In standard practice the peptides will each be resolved during MS analysis so that if the mass/charge profile of peptides from a protein or polypeptide is known, the quantity of the protein spiked into the sample can be used to calculate the quantity of the corresponding peak from the sample, after dilutions from introduction of the reference standard are accounted for.

Yet another aspect of the present disclosure features quantifying the abundance of biological derivatives, for example, biomolecules acted on by an environmental substance, for example a toxic substance. A known derivative may be synthesized, and used to spike one or more samples. A derivative may incorporate component atoms of the environmental substance or may result from interaction of a cell with the environmental substance.

Another aspect of the present disclosure features aiding identification of “ion-pairs” using “High/Low” mass spectrometry analysis. In “High/Low” MS, the user programs the MS instrument to alternate the experimental conditions during electrospray ionization such that the voltage cycles between high voltage settings (which induce fragmentation randomly along the backbone of a peptide) and low voltage settings (which preserve the integrity of the peptide backbone). Thus, during the low voltage settings, the instrument is analyzing the parent ions, and in during high voltage conditions these parent ions are all being fragmented simultaneously. This MS technique allows a user to analyze peptides as they elute from a liquid chromatography separation with a very high duty cycle. Because the “High/Low” technique does not use traditional MS/MS methods where one stage of MS selects a precursor mass, and a second stage of MS analyzes fragment ions generated by a collision cell, computational algorithms are required to decipher the data being generated. The present disclosure could aid the identification of “ion-pairs” that are separated by a set mass difference imparted by the number of heavy isotopes. These “ion-pairs” can assist in differentiating low-abundance signals from the background.

In certain embodiments a copy polypeptide may optionally be conjugated to an affinity tag that can be subsequently be cleaved enzymatically or by a self-cleaving mechanism, e.g., 2A self-cleaving peptide. Inclusion of an affinity tag, such as one or more histidine tags, may allow quick and easy purification of the expressed protein by affinity capture, for example with a metal chelate resin. A tag is not necessary however. Any desired tag including an immune recognized tag may be used. Non-limiting examples of affinity tags that may be used in accordance with the present disclosure include for example, VS, FLAG purification tag, c-myc or a poly-His tag (e.g., HH, HHH, HHHHHH (SEQ ID NO: 45) or any polymer or combination thereof), polyhistidine, hemagglutinin, GST, biotin, GFP, and polycysteine (for example, tetracysteine), etc. The tag may alter the mass of only one peptide, for example a C-terminal peptide. Other peptides will be essentially identical in sequence and chemistry to the native protein, but differ in mass due to the use of the non-naturally present isotope.

The copy polypeptide may be further fragmented to produce multiple fragments for MS. Peptide fragments can be formed by any fragmentation process, for example by biological (such as enzymatic, e.g., tryptic) processes or by chemical processes (e.g., chemical cleavage. Tryptic enzymes and cyanogen bromide are preferred fragmentation instruments. Although a quantity can be assayed using a single peptide fragment confidence increases as more data is available. For example, additional fragments increase confidence that the copy is behaving as the copied molecule in fragmentation and MS and allow for averaging to increase confidence in the absolute values obtained.

Once purified, the biomolecule standard may be quantified by biochemical or spectroscopic methods. The concentration of the biomolecule can be assayed by one of many different commercially available methods, e.g., a Lowry assay, or any method chosen by the user. The resulting biomolecule is an isotopically-labeled biomolecule standard. The standard may then be used for example, as a quantitative internal reference for a sample cell lysate.

The present disclosure includes kits for performing the methods, kits for producing the products of the present disclosure, and/or kits that include one or more copy polypeptides of the present disclosure. In certain embodiments, this disclosure relates to kits comprising, 1) the copy of the polypeptide of interest as disclosed herein, 2) an enzyme capable of N-terminal cleave of lysine and arginine, and/or 3) an enzyme capable of C-terminal cleave of lysine or arginine.

Accordingly, kits of the present disclosure may include any components that may be of assistance in performing such methods or producing such products. By way of non-limiting example, kits in accordance with the present disclosure may include kits for quantifying a biomolecule by mass spectrometry or kits for producing isotopically-labeled biomolecule or protein standards.

Kits in accordance with the present disclosure may include means for or one or more components for synthesizing isotopically-labeled biomolecule (e.g., protein) standards, for example using recombinant expression. By way of non-limiting example, such kits may include one or more components for performing in vitro translation, such as an in vitro peptide synthesis system. In vitro peptide synthesis systems may include for example, an extract selected from the group consisting of a bacterial extract, a eukaryotic extract, a plant extract, a mammalian extract, and an insect cell or arthropod extract, and one or more isotopically-labeled amino acids.

In another example, kits can include media, and isotopically-labeled amino acids for production of standards in cell culture. Kits may also include host cells. Kits may further include a template instruction for synthesizing a biomolecule standard.

Kits according to one aspect of the present disclosure include at least one amino acid containing one or more atomic isotopes that are different from naturally occurring isotopes. Non-limiting examples of amino acids in accordance with the present disclosure include one or more amino acids selected from the group consisting of Arg, Lys, Asp, Glu, Met, Trp, Ser, Thr, Tyr, and Asn. Non-limiting examples of isotopes in accordance with the present disclosure include one or more isotopes selected from the group consisting of ¹⁵N, ¹³C, ¹⁸O, ²H and ³⁴S. In addition to an isotopically-labeled amino acid, in one aspect kits of the present disclosure can include one or more of: a culture medium for in vitro culture; a template instruction for synthesizing a biomolecule standard; a cell lysis reagent; a resin for purifying a biomolecule standard; a protein quantitation reagent; an enzyme or chemical reagent capable of cleaving said biomolecule standard; a host cell; a vector; and a polymerase.

Media provided in a kit for growing cells can include one or more isotopically-labeled amino acids, or the one or more isotopically-labeled amino acids can be provided separately. Media for cell culture for producing MS quantitation standards can be depleted in one or more amino acids, such as one or more amino acids that is provided in the kit in isotopically-labeled form. The kit can also include a cell lysis reagent, such as a buffer or detergent solution and/or one or more enzymes.

In another aspect of kits of the disclosure, in addition to an isotopically-labeled amino acid, kits can include one or more of: an in vitro protein synthesis system; a template instruction for synthesizing a biomolecule standard; a cell lysis reagent; a resin for purifying a biomolecule standard; a protein quantitation reagent; an enzyme or chemical reagent capable of cleaving said biomolecule standard; a host cell; a vector, and a polymerase.

The in vitro protein synthesis system comprises a cell lysate, which can be a prokaryotic or eukaryotic cell lysate, as described herein. The extract can be, for example, a bacterial extract, a eukaryotic extract, a plant cell extract, a mammalian cell extract, or an insect cell extract. The kit can further include at least one buffer for in vitro protein synthesis. A buffer for translation can include one or more salts, buffering compounds, nucleotides, amino acids, enzymes, or energy sources. Preferably, an in vitro synthesis buffer that includes amino acids is depleted in one or more naturally-occurring amino acids that is provided in isotopically-labeled form.

Kits for production of MS protein quantitation standards using in vitro protein synthesis or cell culture systems can further include a resin for purifying a biomolecule standard, such as a protein standard. The resin can be, for example, chromatography media for affinity purification, and can included, without limitation, an Ni-NTA agarose matrix, or a matrix that includes bound maltose, calmodulin, biotin, an antibody, protein A, or other affinity capture reagents. A protein quantitation reagent included in a kit can include, without limitation, a BCA reagent, a Lowry assay reagent, a Bradford assay reagent, an ELISA reagent, or a reagent for fluorometric detection and quantitation.

Kits in accordance with the present disclosure may include an enzyme or chemical preparation capable of cleaving a biomolecule standard. Enzymes in accordance with the present disclosure may be for example, trypsin, nispyrtase, lysarginase, Endo-Lys-C, Endo-Glu-C, AspN protease, yeast peptidase, V-8 protease, pepsin, subtilisin, and tobacco etch virus protease. Chemical preparation in accordance with the present disclosure may include for example, Cyanogen Bromide.

In another aspect, kits in accordance with the present disclosure may include a population of biomolecule standard precursor molecules, which may include at least one atomic isotope that under mass spectrometry produces at least one peak distinguishing the biomolecule standards from otherwise identical biomolecules lacking said at least one atomic isotope. Such kits may further include for example, a catalyst.

According to another aspect of the disclosure, kits may include isotopically-labeled biomolecule standards. The biomolecule standards may include at least one atomic isotope that under mass spectrometry produces at least one peak distinguishing the biomolecule standard from otherwise identical biomolecules lacking the at least one atomic isotope. Kits in accordance with the present disclosure may also include a device for quantifiable spiking or introducing a biomolecule standard to a lysate. The kits can further optionally include a MALDI matrix, such as, for example sinapinic acid or alpha-cyano-4-hydroxycinnamic acid (CHCA). In some embodiments, kits of the disclosure may further comprise mass spectrometric probes.

### EXAMPLES

### Targeted Mass Spectrometry Using Isotopic Heavy Labeled Peptide Standards to Quantify Proteins in Complex Tissues and Biofluids Proteomic Assays

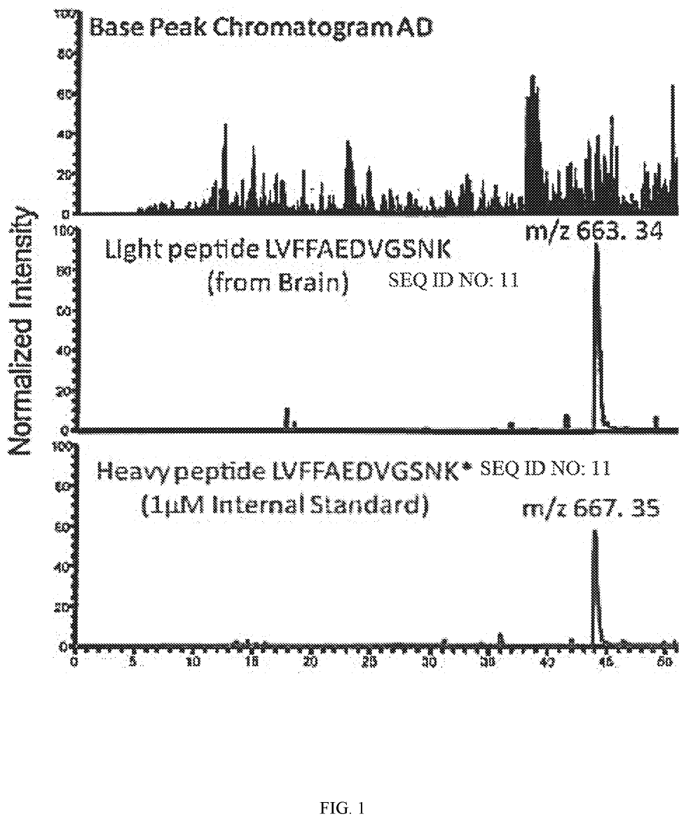

Proteins are first digested into peptides using enzymes such as trypsin. The peptides are then resolved by liquid chromatography (LC) and ionized into the gas phase by electrospray. Precursor peptides are detected in a full scan (MS 1 scan) and subsequently isolated for fragmentation to produce MS/MS scans, which are used to derive sequence specific peptide identifications following database searching. Once unique peptides have been identified for proteins of interest, they can be directly isolated and quantified in “targeted” mass spectrometry assays termed selective reaction monitoring (SRM) or parallel reaction monitoring (PRM), which typically utilize isotope-labeled synthetic peptides or proteins as internal standards to measure the abundance of protein targets in tissue or biofluids. Isotope-labeled peptides are added to each sample and digested with trypsin to produce peptide pairs (light and heavy) that can be resolved by mass spectrometry. For these assays, precursor peptides and product ion pairs are monitored, and their ratio (light/heavy) allows for the relative quantification across samples (e.g. control versus disease). As a quantitative method this provides several advantages: (i) the measurement is highly specific because the corresponding peptides are detected by mass spectrometry directly; (ii) it can measure absolute abundance since isotope-labeled peptides are used at known concentrations; (ii) this strategy circumvents the requirement for antibodies to detect a protein in a complex mixture; and (iv) quantification of hundreds of peptides can be performed in a single sample. The SRM/PRM strategy to quantify specific proteins and post-translational modifications in human brain tissue and cell lines. For example, a targeted mass spectrometry approach was utilized to directly quantify AP from human brain tissue (FIG. 1). Metabolically labeled cells and mouse tissues in lieu of synthetic peptides were used as internal standards, which improves measurement accuracy by accounting for the variants during protein digestion.

### Isotope Labeled Internal Standards for Quantitative Mass Spectrometry Assays

One of the limiting factors for clinical MS based proteomic assays has been the sample preparation required, including batch to batch consistency of proteolytic (e.g. trypsin) digestion used to generate diagnostic peptides. Deficiencies in sample preparation and peptide choice also influence the accuracy and reproducibility of the assays. Trypsin (i.e., TrypsinC) is an enzyme used in bottom-up proteomic approaches. It cleaves C-terminal to lysine (K) and arginine (R). Traditionally, when designing heavy isotope-labeled synthetic peptides for mass spectrometry assays, all peptides will have K or R with ¹³C and ¹⁵N isotopes at the C-terminal end, which are then directly added to the sample following protein digestion.

The premise is that the isotopic heavy-labeled peptides (i.e., AQUA™ peptide) have the same physicochemical properties as their endogenous counterparts, including an identical retention time, ionization efficiency, and MS/MS fragmentation pattern. However, they can be distinguished in a mass spectrometer based on the masses of the precursor and fragment ions when measured by SRM or PRM. However, there are other less utilized enzymes with N-terminal specificity for arginine and lysine that generate complementary peptides to trypsin. These enzymes include Tryp-N™, which is a thermophilic metalloprotease developed at Cold Spring Harbor Laboratory and the thermophilic proteinase LysargiNase isolated from Methanosarcina acetivorans. Therefore, both TrypN™ and LysargiNase effectively mirror trypsin (Tryp-C), to generate complementary peptides from proteins that can be identified and quantified by mass spectrometry. Tryp-C and Tryp-N™ peptides serve as independent or “replicate” measurements that when measured simultaneously within the same assay can reduce the number of analytical replicates needed for high accuracy in biomarker measurements.

Trypsin cleaves C-terminal to lysine (K) and arginine (R). When designing heavy isotope-labeled synthetic peptides for mass spectrometry assays, all peptides will have K or R with ¹³C and ¹⁵N isotopes at the C-terminal end. The premise is that the isotopic heavy-labeled peptides have the same physicochemical properties as their endogenous counterparts, including an identical retention time, ionization efficiency, and MS/MS fragmentation pattern. However, they can be distinguished in a mass spectrometer based on the masses of the precursor (FIG. 1) and fragment ions when measured by SRM or PRM. However, there are other enzymes with N-terminal specificity for arginine and lysine that generate complementary peptides to trypsin. Tryp-N™ is a thermophilic metalloprotease with N-terminal specificity for arginine and lysine. Tryp-N™ provides the complimentary b-ion series that can be missing from trypsin, allowing one to more easily find the N-terminal.

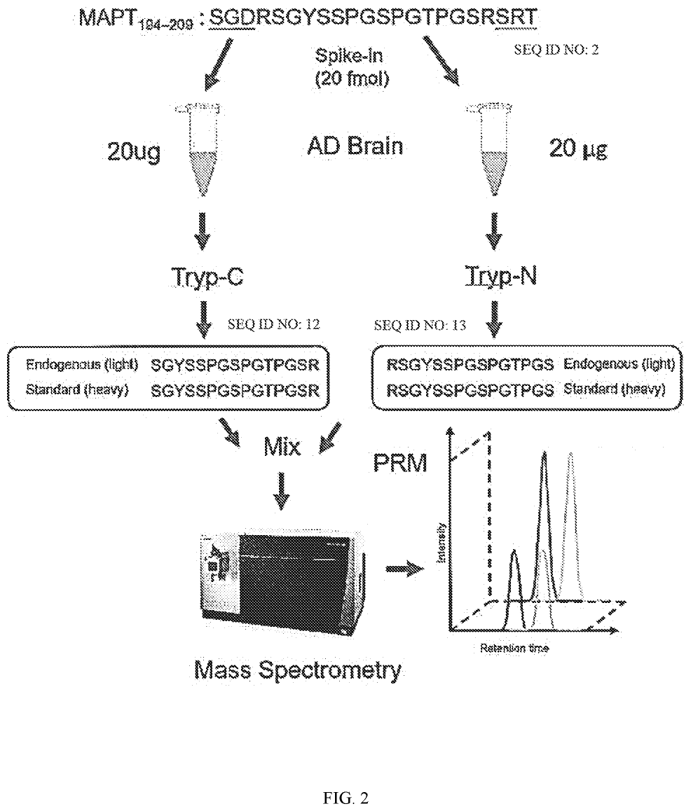

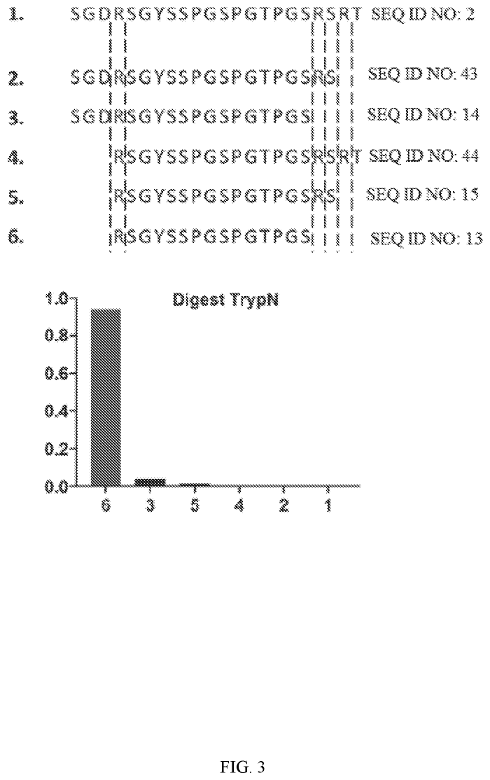

LysargiNase is a thermophilic proteinase isolated from Methanosarcina acetivorans that mirrors trypsin (Tryp-C), to generate complementary peptides from proteins that can be identified and quantified by mass spectrometry. Tryp-C and Tryp-N™ peptides serve as independent or “replicate” measurements that when measured simultaneously within the same assay can reduce the number of analytical replicates needed for high accuracy in biomarker measurements. Isotope labeled peptide standards that are amenable to digestion by both Tryp-C and Tryp-N™. Thus, single isotopically labeled heavy peptide standard was synthesize for Tau SGDRSGYSSPGSPGTPGSRSRT (SEQ ID NO: 2) that harbors a “heavy” arginine (bold) on both the N-terminus and C-terminus corresponding to residues 194-209 as well as flanking residues (underlined) in the full-length Tau protein sequence (FIG. 2). These peptide standards are referred to as CompTryp standards for their amenability for either Tryp-C or Tryp-N™ proteolytic digestion. Tau is the core component of pathological neurofibrillary tangles in Alzheimer's disease (AD) and concurrent with Aβ serves as a key diagnostic biomarker in Alzheimer's disease (AD) in cerebrospinal fluid (CSF). Notably, Tau also serves as a biomarker for other neurodegenerative diseases including corticobasal degeneration (CBD), frontotemporal dementia (FTD) and other diseases collectively termed tauopathies.

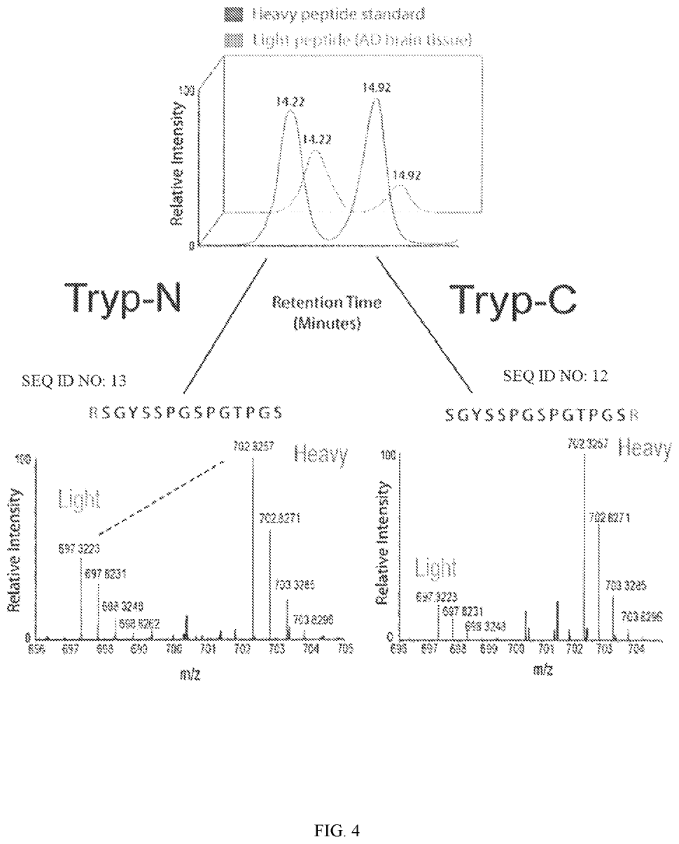

CompTryp standards include flanking amino acid residues adjacent to the Tryp-N™ and Tryp-C cleavage sites to monitor both complete and partially digested peptide intermediates to assess digestion efficiencies. When the Tau CompTryp standard is spiked a known concentration into to AD human brain tissue and digested with Tryp-C, the C-terminal tryptic peptide will be exclusively generated SGYSSPGSPGTPGSR (SEQ ID NO: 12), whereas a separate digestion with Tryp-N™ will generate the N-terminal tryptic peptide RSGYSSPGSPGTPGS (SEQ ID NO: 13) (FIG. 4). TrypC and TrypN™ typically cleave CompTryp standard peptides with 99.8% and 94.1% efficiently, respectively.

Both the Tryp-N™ and Tryp-C heavy peptides have the exact same mass detected in the mass spectrometer (702.32 m/z), yet can be distinguished in the mass spectrometry by retention time by LC and MS/MS scans (FIG. 4). By placing basic residues (K or R) on the amino terminus and side chains, b-ions predominate in MS/MS spectra of Tryp-N™ peptides compared to Tryp-C peptides where y-ions typically dominate. The TrypN™ and TrypC peptides also differ in LC elution times, with the TrypN™ product eluting slightly earlier than the TrypC product. These differences in MS/MS fragmentation and elution time between the TrypN™ and TrypC generated peptide products allows unambiguous quantification of both the light peptide from human CSF and heavy peptide standard by PRM. Collectively these engineered CompTryp heavy peptide standard can produce two unique peptides in parallel digestion strategies for the direct quantification of tau in biofluids.

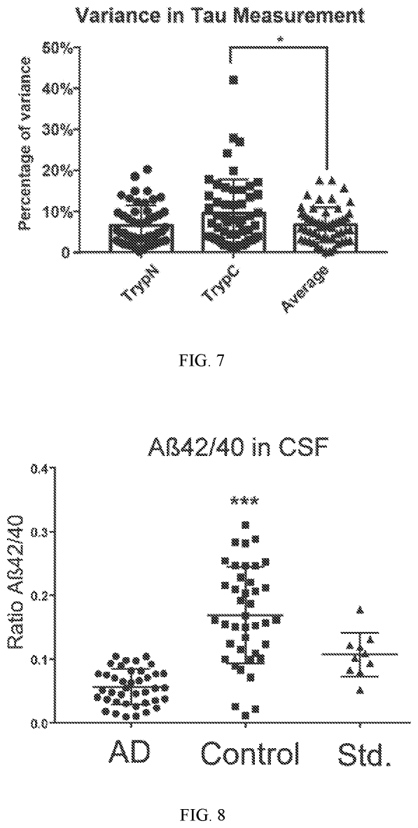

Proteolytic digestion of well-established biomarkers in AD, tau, using two distinct enzymes (Tryp-C and Tryp-N) generates complementary peptide fragments for the direct detection and quantification by MS. By using this complementary tryptic digestion (CompTryp) approach, one can successfully measure two overlapping tau tryptic peptides in CSF. See Wingo et al. Integrating Next-Generation Genomic Sequencing and Mass Spectrometry To Estimate Allele-Specific Protein Abundance in Human Brain. J Proteome Res, 2017. 16(9): p. 3336-3347. Notably, both peptides were highly correlated and equally effective at discriminating patients with AD from patients without AD (FIG. 5A). Furthermore, Tau levels in CSF measured by MS were highly correlated to total Tau level by ELISA (Innotest) from these same samples indicating that the MS-based quantification is comparable to the “gold standard” in biomarker detection (FIG. 5B). Finally, the coefficient of variation (CV) measurements is improved when you average the measurements of TrypN and TrypC PRM value compared to either one alone (FIG. 7).

### Quantification of Tau Levels Using CompTryp Standards in Control and AD Human Brain Tissues

While the quantitation of peptides can be made accurate and precise using peptide standards alone, these measurements do not account for incomplete recovery of peptides during the sample preparation or for the proteolytic digestion of the protein within the biological sample. For example, it is preferable to use protein standards (when available) with known quantity to calibrate the peptide precursor signal in the mass spectrometer. The major advantage of a heavy labeled protein standard is that it undergoes trypsin digestion identical to the endogenous protein. CompTryp standards were engineered to including flanking amino acid residues adjacent to the Tryp-N™ and Tryp-C cleavage site to monitor both complete and partially digested peptide intermediates to assess digestion efficiencies. When the Tau CompTryp standard is spiked a known concentration into to AD human brain tissue and digested with Tryp-C, the C-terminal tryptic peptide will be exclusively generated SGYSSPGSPGTPGSR (SEQ ID NO: 12, whereas a separate digestion with Tryp-N™ will generate the N-terminal tryptic peptide RSGYSSPGSPGTPGS (SEQ ID NO: 13). Remarkably, both the Tryp-N™ and Tryp-C peptides have the exact same mass detected in the mass spectrometer (697.3224 m/z), yet can be distinguished in the mass spectrometry by retention time by LC and MS/MS scans (FIG. 4). Notably, by placing basic residues (K or R) on the amino terminus and side chains, b-ions predominate in MS/MS spectra of Tryp-N™ peptides compared to Tryp-C peptides where y-ions typically dominate.

CompTryp heavy peptide standard can produce two unique peptides in parallel digestion strategies for the direct quantification of Tau in brain or biofluids. Notably, both peptides were equally effective at discriminating patients with AD from controls with signals being significantly higher in control. The light/heavy ratio for the Tryp-C and Tryp-N™ measurements in the same cases were extremely well correlated highlighting the high reproducibility utilizing the complementary digestion approach (FIG. 5).

### Engineering CompTryp Standards for Aβ Measurements by Mass Spectrometry

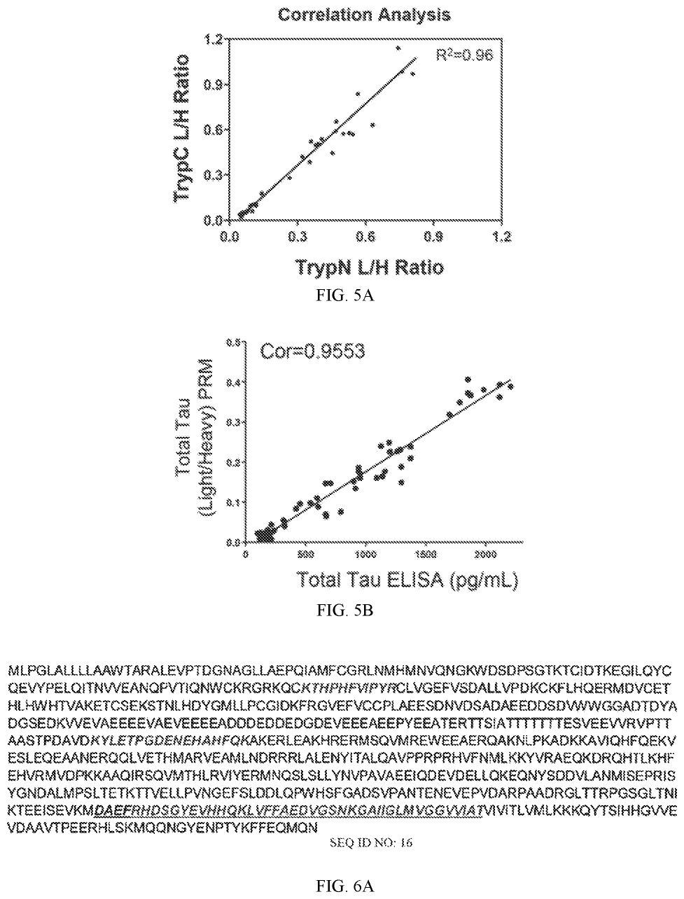

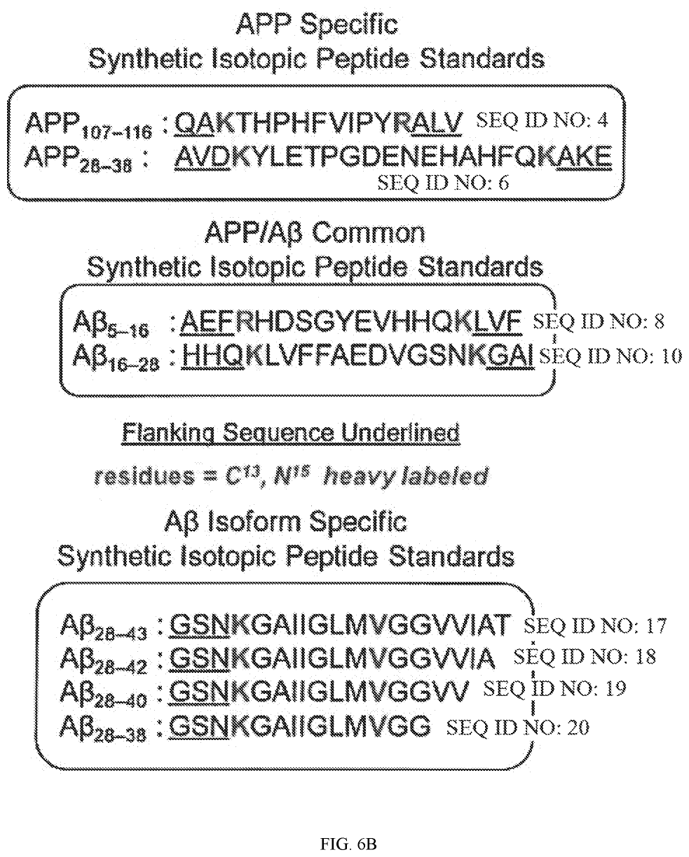

Senile plaques in AD are primarily composed of the peptides Aβ(1-40) and Aβ (1-42), which are cleaved from the amyloid precursor protein (APP) by β-(BACE) and γ-secretases. Of particular importance, the accumulation of Aβ plaques appears to precede the development of cognitive decline by 10 or more years. For this reason, sensitive diagnostic biomarkers have been sought to detect the disease process at an early stage. Based on results for Tau measurements following both Tryp-C and Tryp-N™ cleavage, Aβ CompTryp standards were designed with flanking residues to ensure proteolytic digestion. Typically, intake Aβ isoforms (Aβ 1-40 and Aβ 1-42) in human cerebrospinal fluid (CSF) are analyzed by mass spectrometry using the intact Aβ 1-40 or 1-42 heavy isotope standards. However, given the size of these peptides (about 4 kDa) special chromatography conditions and procedures are needed to ensure that these highly hydrophobic and aggregate prone peptides remain soluble and readily detectable by mass spectrometry, which is not always the case. To overcome these limitations isoform specific CompTryp peptides were developed that can discriminate full length Amyloid Precursor Protein (APP) from A β isoforms (1-38, 1-40, 1-42 and 1-43) utilizing isotopic heavy versions of lysine and valine (FIG. 6B), Following digestion with either Tryp-C or Typ-N™ these isotopic heavy-labeled peptides will have the same physicochemical properties as their endogenous light AP counterparts, including an identical retention time, ionization efficiency, and MS/MS fragmentation pattern. Using these standards one can employ SRM or PRM for targeted quantitation of multiple Aβ isoform peptides due in a single mass spectrometry assay.

Following digestion with Tryp-C or Typ-N™ these isotopic heavy-labeled peptides will have the same physicochemical properties as their endogenous light Aβ peptide counterparts, including an identical retention time, ionization efficiency, and MS/MS fragmentation pattern. As a proof of concept, CompTryp Aβ standards were used to successfully quantify Aβ28-40 and Aβ28-42 peptides in 40 controls and 40 AD patient CSF samples (FIG. 8) by PRM. The peptide ratio of Aβ42/40 was lower in AD and capable of discriminating AD from control CSF samples.

Translating Protein Networks in Brain into Biomarker Panels for AD Diagnostics.

Methods provided herein are broadly applicable to generating peptide surrogates for any brain specific protein biomarker that we reliably detect in CSF. By compiling MS-based proteomic results a comprehensive library was generated of >43,000 peptides corresponding to >4500 proteins that can be detected in CSF, ˜70% of which overlap with proteins in brain modules. As one example, for proof of concept linking protein expression from brain modules to CSF, a MS-based assay was developed using CompTryp standards targeting SMOC1 from an inflammatory community of proteins. In human brain,

SMOC1 is one of the most significantly increased proteins in early asymptomatic AD (AsymAD) and AD brain, and similarly, SMOC1 levels in CSF significantly discriminate AD from controls patients

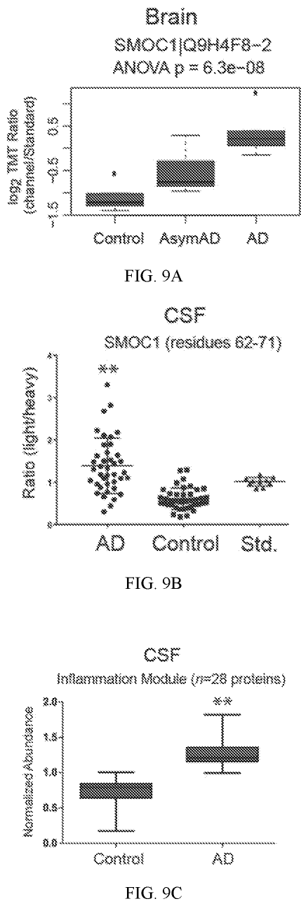

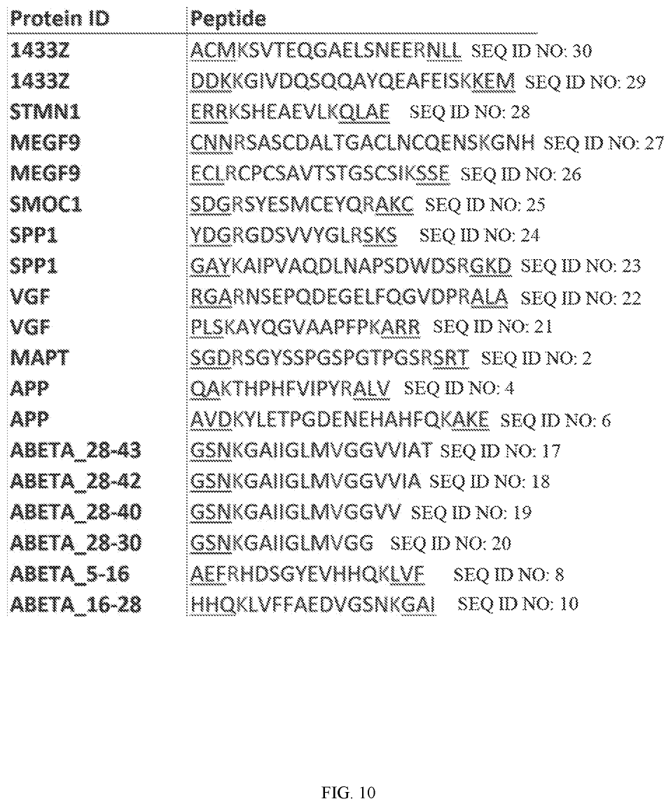

(FIG. 9 A & B). To assess the feasibility of using a panel of proteins to serve as a composite biomarker for a given module, 28 of the most representative proteins were targeted in the same inflammatory brain module by MS, with a single averaged expression metric for the panel strongly discriminating AD from controls (FIG. 9C). Overall, these data demonstrate the ability to target proteins from brain networks enabling an approach for discovery and validation of CSF biomarkers. Using this technology, one can design isotopically heavy labeled CompTryp peptide standards and implement MS assays for their quantification as protein biomarkers in biofluids for the early diagnosis of AD and other chronic brain disorders. FIG. 10 provides a summary of CompTryp peptides that have been synthesized that can be used as a peripheral CSF biomarker panels designed specifically to measure the pathophysiologies that underlie AD in human brain.

What is claimed is:

1. A method for quantifying a polypeptide comprising:
providing a sample having a polypeptide of interest to be quantified;
providing a copy of the polypeptide of interest, the copy comprising two amino acids with isotopes, wherein the two amino acids are two lysine, or two arginine, or a lysine and arginine;
separating the sample into a first sample and a second sample;
introducing a known quantity of the copy into the first sample and the second sample; wherein the copy includes an N-terminal polypeptide linked to a first lysine or a first arginine comprising a first isotope following by a center polypeptide linked to a second lysine or second arginine comprising a second isotope followed by a C-terminal polypeptide;
introducing an enzyme capable of N-terminal cleavage of lysine or arginine into the first sample, providing a N-terminally cleaved peptide with an N-terminal isotopic lysing or arginine;
introducing an enzyme cable of C-terminal cleavage of lysine or arginine into the second sample, providing a C-terminally cleaved peptide with a C-terminal isotopic lysing or arginine;
analyzing the first sample and second sample by mass spectrometry;
comparing obtained mass spectrometry peak pairs resulting from the N-terminally cleaved peptide of the peptide of interest in the first sample, and N-terminally cleaved peptide of the copy to determine a quantity of the peptide of interest in the sample, providing a first quantity of the peptide of interest; and
comparing obtained mass spectrometry peak pairs resulting from the C-terminally cleaved peptide of the peptide of interest in the second sample, and C-terminally cleaved peptide of the copy to determine a quantity of the peptide of interest in the sample, providing a second quantity of the peptide of interest.

2. The method of claim 1, further comprising averaging the first quantity and the second quantity.

3. The method of claim 1 wherein the sample is blood, plasma, brain tissue, or cerebrospinal fluid.

4. The method of claim 1, wherein the copy comprises SEQ ID NO: 1 (RSGYSSPGSPGTPGSR), SEQ ID NO: 3 (KTHPHFVIPYR), SEQ ID NO: 5 (KYLETPGDENEHAHFQK), SEQ ID NO: 7 (RHDSGYEVHHQK), SEQ ID NO: 9 (KLVFFAEDVGSNK), SEQ ID NO: 31 (KGAIIGLMV), SEQ ID NO: 32 (KAYQGVAAPFPK), SEQ ID NO: 33 (RNSEPQDEGELFQGVDPR), SEQ ID NO: 34 (RNSEPQDEGELFQGVDPR), SEQ ID NO: 35 (KAIPVAQDLNAPSDWDSR), SEQ ID NO: 36 (RGDSVVYGLR), SEQ ID NO: 37 (RSYESMCEYQR), SEQ ID NO: 38 (RCPCSAVTSTGSCSIK), SEQ ID NO: 39 (RSASCDALTGACLNCQENSK), SEQ ID NO: 40 (KSHEAEVLK), SEQ ID NO: 41 (KGIVDQSQQAYQEAFEISKK), or SEQ ID NO: 42 (KSVTEQGAELSNEER).

5. The method of claim 1, wherein the copy consist of SEQ ID NO: 2 (SGDRSGYSSPGSPGTPGSRSRT), SEQ ID NO: 4 (QAKTHPHFVIPYRALV), SEQ ID NO: 6 (AVDKYLETPGDENEHAHFQKAKE), SEQ ID NO: 8 (AEFRHDSGYEVHHQKLVF), SEQ ID NO: 10 (HHQKLVFFAEDVGSNKGAI), SEQ ID NO: 17 (GSN KGAIIG LMVGGVVIAT), SEQ ID NO: 18 (GSN KGAIIGLMVGGVVIA), SEQ ID NO: 19 (GSNKGAIIGLMVGGVV), SEQ ID NO: 20 (GSN KGAIIGLMVGG), SEQ ID NO: 21 (PLSKAYQGVAAPFPKARR), SEQ ID NO: 22 (RGARNSEPQDEGELFQGVDPRALA), SEQ ID NO: 23 (GAYKAIPVAQDLNAPSDWDSRGKD), SEQ ID NO: 24 (YDGRGDSVVYGLRSKS), SEQ ID NO: 25 (SDGRSYESMCEYQRAKC), SEQ ID NO: 26 (ECLRCPCSAVTSTGSCSIKSSE), SEQ ID NO: 27 (CNNRSASCDALTGACLNCQENSKGNH), SEQ ID NO: 28 (ERRKSHEAEVLKQLAE), SEQ ID NO: 29 (DDKKGIVDQSQQAYQEAFEISKKEM), or SEQ ID NO: 30 (ACMKSVTEQGAELSNEERNLL).

6. The method of any of claim 1, wherein the first quantity, second quantity, or combination thereof, of the peptide of interest, is used to diagnosed or monitor Alzheimer's disease (AD), corticobasal degeneration (CBD), frontotemporal dementia (FTD), or other neurodegenerative diseases or tauopathies.

7. A polypeptide comprising an N-terminal polypeptide linked to a first lysine or a first arginine comprising a first isotope following by center polypeptide linked to a second lysing or second arginine comprising a second isotope followed by a C-terminal polypeptide.

8. The polypeptide of claim 7 comprising SEQ ID NO: 1 (RSGYSSPGSPGTPGSR), SEQ ID NO: 3 (KTHPHFVIPYR), SEQ ID NO: 5 (KYLETPGDENEHAHFQK), SEQ ID NO: 7 (RHDSGYEVHHQK), SEQ ID NO: 9 (KLVFFAEDVGSNK), SEQ ID NO: 31 (KGAIIGLMV), SEQ ID NO: 32 (KAYQGVAAPFPK), SEQ ID NO: 33 (RNSEPQDEGELFQGVDPR), SEQ ID NO: 34 (RNSEPQDEGELFQGVDP), SEQ ID NO: 35 (KAIPVAQDLNAPSDWDSR), SEQ ID NO: 36 (RGDSVVYGLR), SEQ ID NO: 37 (RSYESMCEYQR), SEQ ID NO: 38 (RCPCSAVTSTGSCSIK), SEQ ID NO: 39 (RSASCDALTGACLNCQENSK), SEQ ID NO: 40 (KSHEAEVLK), SEQ ID NO: 41 (KGIVDQSQQAYQEAFEISKK), or SEQ ID NO: 42 (KSVTEQGAELSNEER).

9. The polypeptide of claim 7, comprising SEQ ID NO: 2 (SGDRSGYSSPGSPGTPGSRSRT), SEQ ID NO: 4 (QAKTHPHFVIPYRALV), SEQ ID NO: 6 (AVDKYLETPGDENEHAHFQKAKE), SEQ ID NO: 8 (AEFRHDSGYEVHHQKLVF), SEQ ID NO: 10 (HHQKLVFFAEDVGSNKGAI), SEQ ID NO: 17 (GSNKGAIIGLMVGGVVIAT), SEQ ID NO: 18 (GSNKGAIIGLMVGGVVIA), SEQ ID NO: 19 (GSNKGAIIGLMVGGVV), SEQ ID NO: 20 (GSN KGAIIGLMVGG), SEQ ID NO: 21 (PLSKAYQGVAAPFPKARR), SEQ ID NO: (RGARNSEPQDEGELFQGVDPRALA), SEQ ID NO: 23 (GAYKAIPVAQDLNAPSDWDSRGKD), SEQ ID NO: 24 (YDGRGDSVVYGLRSKS), SEQ ID NO: 25 (SDGRSYESMCEYQRAKC), SEQ ID NO: 26 (ECLRCPCSAVTSTGSCSIKSSE), SEQ ID NO: 27 (CNNRSASCDALTGACLNCQENSKGNH), SEQ ID NO: 28 (ERRKSHEAEVLKQLAE), SEQ ID NO: 29 (DDKKGIVDQSQQAYQEAFEISKKEM), or SEQ ID NO: 30 (ACMKSVTEQGAELSNEERNLL).

10. A kit comprising:
1) a copy of a polypeptide of interest,
2) an enzyme capable of N-terminal cleavage of lysine and arginine, and
3) an enzyme cable of C-terminal cleavage of lysine or arginine;
wherein the copy comprises an N-terminal polypeptide linked to a first lysine or a first arginine comprising a first isotope following by center polypeptide linked to a second lysing or second arginine comprising a second isotope followed by a C-terminal polypeptide.

11. The kit of claim 10, wherein copy comprises SEQ ID NO: 1 (RSGYSSPGSPGTPGSR), SEQ ID NO: 3 (KTHPHFVIPYR), SEQ ID NO: 5 (KYLETPGDENEHAHFQK), SEQ ID NO: 7 (RHDSGYEVHHQK), SEQ ID NO: 9 (KLVFFAEDVGSNK), SEQ ID NO: 31 (KGAIIGLMV), SEQ ID NO: 32 (KAYQGVAAPFPK), SEQ ID NO: 33 (RNSEPQDEGELFQGVDPR), SEQ ID NO: 34 (RNSEPQDEGELFQGVDPR), SEQ ID NO: 35 (KAIPVAQDLNAPSDWDSR), SEQ ID NO: 36 (RGDSVVYGLR), SEQ ID NO: 37 (RSYESMCEYQR), SEQ ID NO: 38 (RCPCSAVTSTGSCSIK), SEQ ID NO: 39 (RSASCDALTGACLNCQENSK), SEQ ID NO: 40 (KSHEAEVLK), SEQ ID NO: 41 (KGIVDQSQQAYQEAFEISKK), or SEQ ID NO: 42 (KSVTEQGAELSNEER).

12. The kit of claim 10, wherein copy consists of SEQ ID NO: 2 (SGDRSGYSSPGSPGTPGSRSRT), SEQ ID NO: 4 (QAKTHPHFVIPYRALV), SEQ ID NO: 6 (AVDKYLETPGDENEHAHFQKAKE), SEQ ID NO: 8 (AEFRHDSGYEVHHQKLVF), SEQ ID NO: 10 (HHQKLVFFAEDVGSNKGAI), SEQ ID NO: 17 (GSN KGAIIG LMVGGVVIAT), SEQ ID NO: 18 (GSNKGAIIGLMVGGVVIA), SEQ ID NO: 19 (GSNKGAIIGLMVGGVV), SEQ ID NO: 20 (GSN KGAIIGLMVGG), SEQ ID NO: 21 (PLSKAYQGVAAPFPKARR), SEQ ID NO: 22 (RGARNSEPQDEGELFQGVDPRALA), SEQ ID NO: 23 (GAYKAIPVAQDLNAPSDWDSRGKD), SEQ ID NO: 24 (YDGRGDSVVYGLRSKS), SEQ ID NO: 25 (SDGRSYESMCEYQRAKC), SEQ ID NO: 26 (ECLRCPCSAVTSTGSCSIKSSE), SEQ ID NO: 27 (CNNRSASCDALTGACLNCQENSKGNH), SEQ ID NO: 28 (ERRKSHEAEVLKQLAE), SEQ ID NO: 29 (DDKKGIVDQSQQAYQEAFEISKKEM), or SEQ ID NO: 30 (ACMKSVTEQGAELSNEERNLL).
